(19) United States
(12) Patent Application Publication
(10) Pub. No.: US 2010/0233765 A1
(43) Pub. Date: Sep. 16, 2010

(54) L-CYSTEINE-PRODUCING BACTERIUM AND A METHOD FOR PRODUCING L-CYSTEINE

(76) Inventor: Gen Nonaka, Kawasaki-shi (JP)

Correspondence Address: CERMAK NAKAJIMA LLP;ACS LLC, 127 S. Peyton Street, Suite 210, ALEXANDRIA, VA, 22314, US

(21) Appl. No.: 12/722,094

(22) Filed: Mar. 11, 2010

(30) Foreign Application Priority Data
Mar. 12, 2009 (JP) 2009-059792

(51) Int. Cl. C12P 13/12; C12N 1/21

(52) U.S. Cl. 435/113; 435/252.3; 435/252.33

(57) ABSTRACT

The present invention provides a bacterium belonging to the family Enterobacteriaceae, which is able to produce L-cysteine, and has been modified to decrease activity of the YdjN protein, or the activities of the YdjN and the FliY protein. This bacterium is cultured in a medium, and L-cysteine, L-cystine, a derivative or precursor thereof, or a mixture of these can be collected from the medium.

This application claims priority under 35 U.S.C. §119 to Japanese Patent Application No. 2009-059792, filed on Mar. 12, 2009, which are incorporated in their entireties by reference. The Sequence Listing in electronic format filed herewith is also hereby incorporated by reference in its entirety (File Name: 2010-3-11T_US-426_Seq_List; File Size: 113 KB; Date Created: Mar. 11, 2010).

### BACKGROUND OF THE INVENTION

1. Field of the Invention

The present invention relates to a method for producing L-cysteine and related substances. Specifically, the present invention relates to a bacterium suitable for producing L-cysteine and related substances and a method for producing L-cysteine and related substances utilizing the bacterium. L-cysteine and L-cysteine-related substances are useful in the fields of drugs, cosmetics, and food.

2. Brief Description of the Related Art

L-cysteine can be obtained by extraction from keratin-containing substances such as hair, horns and feathers, or by the conversion of the precursor DL-2-aminothiazoline-4-carboxylic acid using a microbial enzyme. L-cysteine has also been produced in a large scale by an immobilized enzyme method utilizing a novel enzyme.

Furthermore, L-cysteine has also been produced by fermentation utilizing a bacterium. For example, a method has been disclosed for producing L-cysteine using an Escherichia bacterium having a suppressed L-cysteine decomposition system and a serine acetyltransferase (EC 2.3.1.30, henceforth also referred to as “SAT”) in which feedback inhibition by L-cysteine is attenuated (Japanese Patent Laid-open (Kokai) No. 11-155571). Furthermore, as bacteria with enhanced L-cysteine-producing ability via suppression of the L-cysteine decomposition system include coryneform bacteria or Escherichia bacteria in which activity of cystathionine-β-lyase (Japanese Patent Laid-open No. 11-155571), tryptophanase (Japanese Patent Laid-open No. 2003-169668), or O-acetylserine sulfhydrylase B (Japanese Patent Laid-open No. 2005-245311) is attenuated or deleted. A method for producing L-cysteine by using a bacterium in which L-cysteine metabolism is decontrolled by using a DNA sequence coding for SAT that has a specific mutation for attenuating feedback inhibition by L-cysteine is also known (National Publication of Translated Version in Japan (Kohyo) No. 2000-504926).

Furthermore, the ydeD gene which encodes the YdeD protein has been reported (Dabler et al., Mol. Microbiol., 36, 1101-1112 (2000)). Also, the yfiK gene that encodes the YfiK protein (Japanese Patent Laid-open No. 2004-49237) participates in secretion of the metabolic products of the cysteine pathway. Furthermore, techniques are known for enhancing L-cysteine-producing ability by increasing expression of the mar-locus, acr-locus, cmr-locus, mex-gene, bmr-gene or qacA-gene, each of which encode proteins suitable for secreting a toxic substance from cells (U.S. Pat. No. 5,972,663), or emrAB, emrKY, yojlH, acrEF, bcr or cusA gene (Japanese Patent Laid-open No. 2005-287333).

As an L-cysteine-producing bacterium, Escherichia coli in which the activity of the positive transcription control factor of the cysteine regulon encoded by the cysB gene is increased has been reported (International Patent Publication WO01/27307).

Furthermore, in the production of L-amino acids by fermentation, not only is secretion of L-amino acids out of cells important, but also uptake of L-amino acids. Microorganisms are able to take up many kinds of amino acids into the cells from the environmen, in which the microorganism grows, and use them. For example, Escherichia coli (E. coli) has a large number of transporters, and it is supposed that many of them participate in uptake of L-amino acids. It has even been estimated that among the predicted “membrane tranporter proteins”, 14% participate in transport of amino acids (Paulsen et al., J. Mol. Biol., 277, 573-592 (1998)). However, a large number of various transporter paralogues relate to substrate specificity, and there are overlapping functions, such as plural uptake systems for the same substrate. Therefore, it is extremely difficult to identify transporter function (Hosie et al., Res. Microbiol., 152, 259-270 (2001)). As described above, functions and physiological roles of transporters are very complicated. Therefore, when the characteristics of a transporter are simply estimated on the basis of homology or phenotype alone, these characteristics may not adequately reflect physiological functions as an actual transporter. For example, if a particular substrate is transported and this is a physiologically important function, there may be other substrates which are also transported, or the like. Furthermore, even if a certain substance is found to be transported, there may be plural factors which also transport that substance. Therefore, when a microorganism is modified to produce amino acids by fermentation, it is not easy to target a transporter for modification.

Furthermore, although there are several reports, as described below, of uptake systems of bacteria for cystine, there have been no substantial findings reported about the uptake of L-cysteine and S-sulfocysteine.

It is expected that E. coli has at least two kinds of cystine uptake systems having different kinetic characteristics (Berger et al., J. Biol. Chem., 247, 7684-7694 (1972)). FliY has been demonstrated to bind to cystine in an in vitro experimental system (Butler et al., Life Sci., 52, 1209-1215 (1993)), and the fliY gene is expected to form an operon with yecC, yecS and yecO, which are located nearby, and function as an ABC transporter (Hosie et al., Res. Microbiol., 152, 259-270 (2001)). However, it has not been experimentally demonstrated yet whether they function as a physiological cystine uptake system in E. coli.

Although it is similarly expected that three cystine uptake systems with different kinetics are also present in Salmonella bacteria (Baptist et al., J. Bacteriol., 131, 111-118 (1977)), the involved proteins and genes coding for them have not been identified yet. Furthermore, three kinds of cystine uptake systems (YckKJI, YtmJKLMN, YhcL) have been reported for Bacillus subtilis, and if these three systems are deleted, the bacterium is unable to grow with cystine as the sole sulfur source (Burguiere et al., J. Bacteriol., 186, 4875-4884 (2004)).

Although it has been reported that YdjN of E. coli has a homology of 45% to TcyP, which is known to be involved in cystine uptake of Bacillus subtilis (Burguiere et al., J. Bacteriol., 186, 4875-4884 (2004)), it has not been confirmed whether it actually has cystine uptake activity.

It is known that in Lactobacillus fermentum BR11, bspA codes for a cystine uptake system (Turner et al., J. Bacteriol., 181, 2192-2198 (1999)). Furthermore, although it is expected that there are two cysteine uptake systems with different kinetics in Legionella pneumophila (Ewann et al., Appl. Environ. Microbiol., 72, 3993-4000 (2006)), neither the involved genes nor proteins have been identified yet.

### SUMMARY OF THE INVENTION

Aspects of the present invention include developing novel techniques for improving bacterial production of L-cysteine, and thereby providing an L-cysteine-producing bacterium, as well as a method for producing L-cysteine, L-cystine, a derivative or precursor thereof, or a mixture of these using such a bacterium.

These aspects were achieved by finding that the ability of a bacterium could be improved by modifying the bacterium to decrease activity of the protein encoded by the ydjN gene, and the ability to produce L-cysteine could be further improved by modifying the bacterium to decrease the activity of a protein encoded by the fliY gene, in addition to the foregoing protein.

It is an aspect of the present invention to provide a bacterium belonging to the family Enterobacteriaceae, which is able to produce L-cysteine, and has been modified to decrease the activity of the YdjN protein.

It is a further aspect of the present invention to provide the bacterium as described above, wherein the YdjN protein has the amino acid sequence of SEQ ID NO: 2 or 4, or a variant thereof.

It is a further aspect of the present invention to provide the bacterium as described above, which has been further modified to decrease the activity of the FliY protein.

It is a further aspect of the present invention to provide the bacterium as described above, wherein the FliY protein has the amino acid sequence of SEQ ID NO: 6 or 8, or a variant thereof.

It is a further aspect of the present invention to provide the bacterium as described above, wherein the activity of the YdjN or FliY protein is decreased by a method selected from the group consisting of A) reducing expression of the ydjN or fliY gene, B) disrupting the ydjN or fliY gene, and combinations thereof.

It is a further aspect of the present invention to provide the bacterium as described above, wherein the ydjN gene is selected from the group consisting of:

(a) a DNA comprising the nucleotide sequence of SEQ ID NO: 1 or 3,

(b) a DNA which is able to hybridize with a sequence complementary to the nucleotide sequence of SEQ ID NO: 1 or 3, or a probe which is prepared from the nucleotide sequence under stringent conditions, and

(c) a DNA which has a homology of 95% or more to the nucleotide sequence of SEQ ID NO: 1 or 3.

It is a further aspect of the present invention to provide the bacterium as described above, wherein the fliY gene is selected from the group consisting of:

(d) a DNA comprising the nucleotide sequence of SEQ ID NO: 5 or 7,

(e) a DNA which is able to hybridize with a sequence complementary to the nucleotide sequence of SEQ ID NO: 5 or 7, or a probe which is prepared from the nucleotide sequence, under stringent conditions, and

(f) a DNA which has a homology of 95% or more to the nucleotide sequence of SEQ ID NO: 5 or 7.

It is a further aspect of the present invention to provide the bacterium as described above, which further has at least one of the following characteristics:

i) it has been modified to increase serine acetyltransferase activity,

ii) it has been modified to increase expression of the yeaS gene,

iii) it has been modified to increase 3-phosphoglycerate dehydrogenase activity,

iv) it has been modified to enhance activity of the sulfate/thio sulfate transport system.

It is a further aspect of the present invention to provide the bacterium as described above, which is belongs to the genus Pantoea.

It is a further aspect of the present invention to provide the bacterium as described above, which is Pantoea ananatis.

It is a further aspect of the present invention to provide the bacterium as described above, which is Escherichia coli.

It is a further aspect of the present invention to provide a method for producing a product selected from the group consisting of L-cysteine, L-cystine, a derivative or precursor thereof, and combinations thereof, which comprises culturing the bacterium as described above in a medium and collecting the product from the medium.

According to the present invention, L-cysteine-producing ability of bacteria belonging to the family Enterobacteriaceae can be improved. Furthermore, according to the present invention, L-cysteine, L-cystine, derivatives and precursors thereof, and mixtures of them can be efficiently produced.

Still other aspects, features, and attendant advantages of the present invention will become apparent to those skilled in the art from a reading of the following detailed description of embodiments constructed in accordance therewith, taken in conjunction with the accompanying drawings.

Still other objects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

FIG. 1 shows uptake of S-sulfocysteine by E. coli MG1655.

FIG. 2 shows uptake of cystine by E. coli MG1655.

FIG. 3 shows uptake of cysteine by E. coli MG1655.

FIG. 4 shows uptake of S-sulfocysteine by P. ananatis ydjN.

FIG. 5 shows uptake of cystine byfliY-deficient E. coli.

FIG. 6 shows uptake of cystine byfliY-enhanced E. coli.

FIG. 7 shows uptake of cysteine byfliY-deficient E. coli.

FIG. 8 shows the sequence of the promoter Pnlp (SEQ ID NO: 62).

### DETAILED DESCRIPTION OF EXEMPLARY EMBODIMENTS

### <1> Bacterium

The bacterium in accordance with the presently disclosed subject matter belongs to the family Enterobacteriaceae, is able to produce L-cysteine, and has been modified to decrease activity of the YdjN protein. An exemplary embodiment of the bacterium is the bacterium as described above, which has been further modified to decrease the activity of the FliY protein in addition to the YdjN protein. The YdjN and FliY proteins are encoded by the fliY and ydjN genes, respectively. These proteins and genes will be explained later.

The L-cysteine-producing ability can refer to an ability of the bacterium to produce L-cysteine in a medium or cells and cause accumulation of L-cysteine in such an amount that L-cysteine can be collected from the medium or cells when the bacterium is cultured in the medium. Furthermore, a bacterium having L-cysteine-producing ability can mean a bacterium which can produce and cause accumulation of a larger amount L-cysteine in a medium or cells as compared with a wild-type, parent, or unmodified strain, and can mean a microorganism which can produce and cause accumulation of L-cysteine in a medium in an amount of, for example, 0.3 g/L or more, 0.4 g/L or more, or even 0.5 g/L or more.

A portion of the L-cysteine produced by the microorganism can be converted into L-cystine in the medium by the formation of a disulfide bond. Furthermore, as described below, S-sulfocysteine may be generated by the reaction of L-cysteine and thio sulfuric acid which are present in the medium (Szczepkowski T. W., Nature, vol. 182 (1958)). Furthermore, L-cysteine generated in bacterial cells may be condensed with a ketone, aldehyde, or, for example, pyruvic acid, which is present in the cells, to produce a thiazolidine derivative via a hemithioketal (refer to Japanese Patent No. 2992010). Thiazolidine derivative and hemithioketal can exist as an equilibrated mixture. Therefore, the ability to produce L-cysteine is not limited to the production of only L-cysteine in a medium or cells, but also includes the production of L-cystine or a derivative or precursor thereof, or a mixture of these, in addition to L-cysteine. Examples of the aforementioned derivative of L-cysteine or L-cystine include, for example, S-sulfocysteine, thiazolidine derivatives, hemithioketals, and so forth. Examples of the precursor of L-cysteine or L-cystine include, for example, O-acetylserine, which is a precursor of L-cysteine. The precursors of L-cysteine or L-cystine also include derivatives of the precursors, and examples include, for example, N-acetylserine, which is a derivative of O-acetylserine, and so forth.

O-Acetylserine (OAS) is a precursor of L-cysteine biosynthesis. OAS is a metabolite of bacteria and plants, and is produced by acetylation of L-serine induced as an enzymatic reaction catalyzed by serine acetyltransferase (SAT). OAS is further converted into L-cysteine in cells.

The ability to produce L-cysteine can be inherent to the bacterium, or it may be obtained by modifying a microorganism such as those described below by mutagenesis or a recombinant DNA technique. In the present invention, unless specially mentioned, the term L-cysteine may be used to refer to reduced type L-cysteine, L-cystine, a derivative or precursor such as those mentioned above or a mixture thereof.

The bacterium is not particularly limited so long as the bacterium belongs to the family Enterobacteriaceae such as those of the genera Escherichia, Enterobacter, Pantoea, Klebsiella, Serratia, Erwinia, Salmonella and Morganella, and has L-cysteine-producing ability. Specifically, those classified into the family Enterobacteriaceae according to the taxonomy used in the NCBI (National Center for Biotechnology Information) database (http://www.ncbi.nlm.nih.gov/Taxonomy/Browser/wwwtax.cgi?id=91347) can be used. As the parent strain of the family Enterobacteriaceae, a bacterium of the genus Escherichia, Enterobacter, Pantoea, Erwinia, or Klebsiella can be used.

Although the Escherichia bacteria are not particularly limited, specifically, those described in the work of Neidhardt et al. (Backmann B. J., 1996, Derivations and Genotypes of some mutant derivatives of Escherichia coli K-12, p. 2460-2488, Table 1, In F. D. Neidhardt (ed.), Escherichia coli and Salmonella Cellular and Molecular Biology/Second Edition, American Society for Microbiology Press, Washington, D.C.) can be used. Escherichia coli is an example. Examples of Escherichia coli include bacteria derived from the prototype wild-type strain, K12 strain, such as Escherichia coli W3110 (ATCC 27325), Escherichia coli MG1655 (ATCC 47076) and so forth.

These strains are available from, for example, the American Type Culture Collection (Address: P.O. Box 1549, Manassas, Va. 20108, United States of America). That is, registration numbers are given to each of the strains, and the strains can be ordered by using these registration numbers (refer to http://www.atcc.org/). The registration numbers of the strains are listed in the catalogue of the American Type Culture Collection.

Examples of the Enterobacter bacteria include Enterobacter agglomerans, Enterobacter aerogenes and so forth, and examples of the Pantoea bacteria include Pantoea ananatis. Some strains of Enterobacter agglomerans were recently reclassified into Pantoea agglomerans, Pantoea ananatis, or Pantoea stewartii on the basis of nucleotide sequence analysis of 16S rRNA etc. A bacterium belonging to the genus Enterobacter or Pantoea may be used so long as it is classified into the family Enterobacteriaceae.

In particular, Pantoea bacteria, Erwinia bacteria, and Enterobacter bacteria are classified as γ-proteobacteria, and they are taxonomically very close to one another (J. Gen. Appl. Microbiol., 1997, 43, 355-361; International Journal of Systematic Bacteriology, October 1997, pp. 1061-1067). In recent years, some bacteria belonging to the genus Enterobacter were reclassified as Pantoea agglomerans, Pantoea dispersa, or the like, on the basis of DNA-DNA hybridization experiments etc. (International Journal of Systematic Bacteriology, July 1989, 39(3), pp. 337-345). Furthermore, some bacteria belonging to the genus Erwinia were reclassified as Pantoea ananas or Pantoea stewartii (refer to International Journal of Systematic Bacteriology, January 1993; 43(1), pp. 162-173).

Examples of the Enterobacter bacteria include, but are not limited to, Enterobacter agglomerans, Enterobacter aerogenes, and so forth. Specifically, the strains exemplified in European Patent Publication No. 952221 can be used. A typical strain of the genus Enterobacter is the Enterobacter agglomeranses ATCC 12287 strain.

Typical strains of the Pantoea bacteria include, but are not limited to, Pantoea ananatis, Pantoea stewartii, Pantoea agglomerans, and Pantoea citrea. Specific examples of Pantoea ananatis include the Pantoea ananatis AJ13355 strain, SC17 strain, and SC17(0) strain. The SC17 strain was selected as a low phlegm-producing mutant strain from the AJ13355 strain (FERM BP-6614) isolated from soil in Iwata-shi, Shizuoka-ken, Japan as a strain that can proliferate in a low pH medium containing L-glutamic acid and a carbon source (U.S. Pat. No. 6,596,517). The SC17(0) strain was constructed to be resistant to the λ Red gene product for performing gene disruption in Pantoea ananatis (WO2008/075483). The SC17 strain was deposited at the National Institute of Advanced Industrial Science and Technology, International Patent Organism Depository (address: Tsukuba Central 6, 1-1, Higashi 1-Chome, Tsukuba-shi, Ibaraki-ken, 305-8566, Japan) on Feb. 4, 2009, and assigned an accession number of FERM ABP-11091. The SC17(0) strain was deposited at the Russian National Collection of Industrial Microorganisms (VKPM), GNII Genetika (address: Russia, 117545 Moscow, 1 Dorozhny proezd. 1) on Sep. 21, 2005 with an accession number of VKPM B-9246.

The Pantoea ananatis AJ13355 strain was deposited at the National Institute of Bioscience and Human-Technology, Agency of Industrial Science and Technology, Ministry of International Trade and Industry (currently, the National Institute of Advanced Industrial Science and Technology, International Patent Organism Depositary, Address: Tsukuba Central 6, 1-1, Higashi 1-Chome, Tsukuba-shi, Ibaraki-ken, 305-8566, Japan) on Feb. 19, 1998 and assigned an accession number of FERM P-16644. It was then converted to an international deposit under the provisions of Budapest Treaty on Jan. 11, 1999 and assigned an accession number of FERM BP-6614. This strain was identified as Enterobacter agglomerans when it was isolated and deposited as the Enterobacter agglomerans AJ13355 strain. However, it was recently reclassified as Pantoea ananatis on the basis of nucleotide sequencing of 16S rRNA and so forth.

Examples of the Erwinia bacteria include, but are not limited to, Erwinia amylovora and Erwinia carotovora, and examples of the Klebsiella bacteria include Klebsiella planticola.

Impartation or Enhancement of L-cysteine-Producing Ability

Hereinafter, methods for imparting L-cysteine-producing ability to bacteria belonging to Enterobacteriaceae, or methods for enhancing L-cysteine-producing ability of such bacteria, are described.

To impart the ability to produce L-cysteine, methods conventionally employed in the breeding of coryneform bacteria or bacteria of the genus Escherichia (see “Amino Acid Fermentation”, Gakkai Shuppan Center (Ltd.), 1st Edition, published May 30, 1986, pp. 77-100) can be used. Such methods include by acquiring the properties of an auxotrophic mutant, an analogue-resistant strain, or a metabolic regulation mutant, or by constructing a recombinant strain so that it overexpresses L-cysteine biosynthesis enzyme. Here, in the breeding of an L-cysteine-producing bacteria, one or more of the above described properties such as auxotrophy, analogue resistance, and metabolic regulation mutation may be imparted. The expression of L-cysteine biosynthesis enzyme(s) can be enhanced alone or in combinations of two or more. Furthermore, the methods of imparting properties such as an auxotrophy, analogue resistance, or metabolic regulation mutation may be combined with enhancement of the biosynthesis enzymes.

An auxotrophic mutant strain, L-cysteine analogue-resistant strain, or metabolic regulation mutant strain with the ability to produce L-cysteine can be obtained by subjecting a parent strain or wild-type strain to conventional mutatagenesis, such as exposure to X-rays or UV irradiation, or treatment with a mutagen such as N-methyl-N′-nitro-N-nitrosoguanidine, ethyl methanesulfonate (EMS) etc., and then selecting those which exhibit autotrophy, analogue resistance, or a metabolic regulation mutation and which also have the ability to produce L-cysteine from the obtained mutant strains.

L-Cysteine-producing ability of a bacterium can be improved by enhancing activity of an enzyme of the L-cysteine biosynthesis pathway or an enzyme involved in production of a compound serving as a substrate of that pathway such as L-serine, for example, 3-phosphoglycerate dehydrogenase, serine acetyltransferase, and so forth. 3-Phosphoglycerate dehydrogenase is subject to feedback inhibition by serine, and therefore the enzymatic activity thereof can be enhanced by incorporating a mutant serA gene coding for a mutant 3-phosphoglycerate dehydrogenase for which the feedback inhibition is eliminated or attenuated into a bacterium.

Furthermore, serine acetyltransferase is subject to feedback inhibition by L-cysteine. Therefore, the enzymatic activity can be enhanced by incorporating a mutant cysE gene coding for a mutant serine acetyltransferase for which the feedback inhibition is eliminated or attenuated into a bacterium.

The L-cysteine-producing ability can also be improved by enhancing the activity of the sulfate/thio sulfate transport system. The sulfate/thio sulfate transport system protein group is encoded by the cysPTWAM gene cluster (Japanese Patent Laid-open No. 2005-137369, European Patent No. 1528108).

The L-cysteine-producing ability of a bacterium can also be improved by increasing expression of the yeaS gene (European Patent Laid-open No. 1016710). The nucleotide sequence of the yeaS gene and the amino acid sequence encoded by the gene are shown in SEQ ID NOS: 15 and 16, respectively. It is known that bacteria use various codons such as GTG, besides ATG, as the start codon (http://depts.washington.edu/agro/genomes/students/stanstart.htm). Although the amino acid corresponding to the initial codon gtg is indicated as Val in SEQ ID NOS: 15 and 16, it is highly possible that it is actually Met.

Specific examples of L-cysteine-producing bacteria include, but not limited to, E. coli JM15 transformed with multiple kinds of cysE gene alleles encoding serine acetyltransferase (SAT) resistant to feedback inhibition (U.S. Pat. No. 6,218,168), E. coli W3110 in which a gene encoding a protein responsible for excretion of cytotoxic substances is overexpressed (U.S. Pat. No. 5,972,663), E. coli strain having decreased cysteine desulfhydrase activity (Japanese Patent Laid-open No. 11-155571), and E. coli W3110 in which activity of the positive transcriptional control factor of the cysteine regulon encoded by the cysB gene is increased (WO01/27307).

For E. coli, proteins are known which have an activity of secreting L-cysteine, such as the protein encoded by ydeD (Japanese Patent Laid-open No. 2002-233384), the protein encoded by yfiK (Japanese Patent Laid-open No. 2004-49237) and the proteins encoded by emrAB, emrKY, yojlH, acrEF, bcr, and cusA, respectively (Japanese Patent Laid-open No. 2005-287333) as described above. Activities of these L-cysteine secreting proteins can be increased.

Hereafter, as the method for imparting an ability to produce L-cysteine, enhancing an activity of L-cysteine biosynthesis system enzyme is described.

Examples of the L-cysteine biosynthesis enzyme include, for example, serine acetyltransferase (SAT). The SAT activity in cells of a bacterium belonging to the family Enterobacteriaceae can be enhanced by increasing the copy number of a gene coding for SAT, or modifying an expression control sequence such as promoter of the gene coding for SAT. For example, a recombinant DNA can be prepared by ligating a gene fragment coding for SAT with a vector, such as a multi-copy vector, which is able to function in the chosen host bacterium belonging to the family Enterobacteriaceae to prepare a recombinant DNA. This recombinant DNA can then be introduced into a host bacterium belonging to the family Enterobacteriaceae to transform it.

Methods for enhancing expression of the SAT gene will be described below. Similar methods can also be applied to other L-cysteine biosynthesis systems enzyme genes, the yeaS gene, and genes of proteins having cysteine secretion activity.

To enhance expression of the SAT gene, modifications can be made, such as, for example, increasing the copy number of the SAT gene in the cells by means of genetic recombination techniques. For example, a recombinant DNA can be prepared by ligating a DNA fragment containing the SAT gene with a vector, such as a multi-copy vector, which is able to function in a host bacterium, and transforming a bacterium with it.

For example, the SAT gene of Escherichia coli can be obtained by PCR using chromosomal DNA of Escherichia coli as a template and primers prepared on the basis of the nucleotide sequence of SEQ ID NO: 9. The SAT genes of other bacteria can also be obtained from chromosomal DNA or a chromosomal DNA library of the bacteria by hybridization using a probe prepared on the basis of the aforementioned sequence information.

The copy number of the SAT gene can also be increased by introducing multiple copies of the SAT gene into a chromosomal DNA of the bacterium. To introduce multiple copies of the SAT gene into a chromosomal DNA of the bacterium, homologous recombination can be performed by targeting a sequence present on the chromosomal DNA in a multiple copy number. A repetitive DNA or inverted repeat present at the end of a transposable element can be used as the sequence present on a chromosomal DNA in a multiple copy number. Alternatively, as disclosed in Japanese Patent Laid-open No. 2-109985, multiple copies of the SAT gene can be introduced into a chromosomal DNA by incorporating them into a transposon and transferring it.

Furthermore, besides amplifying the copy number of a gene described above, expression of the SAT gene can also be enhanced by replacing an expression regulatory sequence of the SAT gene such as a promoter on a chromosomal DNA or a plasmid with a stronger promoter, by amplifying a regulator which increases expression of the SAT gene, or by deleting or attenuating a regulator which reduces expression of the SAT gene. As strong promoters, for example, lac promoter, trp promoter, trc promoter and so forth are known. Furthermore, a promoter of the SAT gene can also be modified to be stronger by introducing substitution of nucleotides or the like into the promoter region of the SAT gene. The aforementioned substitution or modification of the promoter enhances expression of the SAT gene. Examples of methods for evaluating strength of promoters and strong promoters are described in an article by Goldstein and Doi (Goldstein, M. A. and Doi R. H., 1995, Prokaryotic promoters in biotechnology, Biotechnol. Annu. Rev., 1, 105-128), and so forth. Modification of an expression regulatory sequence can be combined with the increasing copy number of the SAT gene. Furthermore, in order to enhance production of the SAT protein, a mutation can be introduced near the translation initiation site of the SAT gene to increase translation efficiency, and this can be combined with enhancement of expression of the SAT gene.

Increase of expression of the SAT gene and increase of the SAT protein amount can be confirmed by quantifying mRNA or by Western blotting using an antibody, as the confirmation of decrease in transcription amount of a target gene and the confirmation of decrease in a target protein amount described later.

As the SAT gene, an SAT gene derived from Escherichia bacteria or an SAT gene derived from other organisms can be used. As the gene coding for SAT of Escherichia coli, cycE has been cloned from a wild-type strain and an L-cysteine excretion mutant strain, and the nucleotide sequence thereof has been elucidated (Denk, D. and Boeck, A., J. General Microbiol., 133, 515-525 (1987)). The nucleotide sequence thereof and the amino acid sequence encoded by the nucleotide sequence are shown in SEQ ID NOS: 9 and 10, respectively. A SAT gene can be obtained by PCR utilizing primers prepared based on the nucleotide sequence and chromosomal DNA of Escherichia bacterium as the template (refer to Japanese Patent Laid-open No. 11-155571). Genes coding for SAT of other organisms can also be obtained in a similar manner. Expression of the SAT gene as described above can be enhanced in the same manner as that for the cysE gene explained above.

When a suppression mechanism such as “feedback inhibition by L-cysteine” exists for the expression of the SAT gene, expression of the SAT gene can also be enhanced by modifying an expression regulatory sequence or a gene involved in the suppression so that the expression of the SAT gene is insensitive to the suppression mechanism.

For example, the SAT activity can be further increased by mutating the SAT so that the feedback inhibition by L-cysteine is reduced or eliminated in the bacterium (henceforth also referred to as “mutant SAT”). Examples of the mutant SAT include SAT having a mutation replacing an amino acid residue corresponding to the methionine residue at position 256 of a wild-type SAT (SEQ ID NO: 10) with an amino acid residue other than lysine residue and leucine residue, or a mutation deleting a C-terminus side region from an amino acid residue corresponding to the methionine residue as position 256. Examples of the amino acid residues other than lysine and leucine include the 17 amino acid residues which typically make up proteins except for methionine, lysine and leucine. Isoleucine and glutamic acid are further examples. To introduce a desired mutation into a wild-type SAT gene, site-specific mutagenesis can be used. As a mutant SAT gene, a mutant cysE coding for a mutant SAT of Escherichia coli is known (refer to International Patent Publication WO97/15673 and Japanese Patent Laid-open No. 11-155571). Escherichia coli JM39-8 strain harboring a plasmid pCEM256E containing a mutant cysE coding for a mutant SAT in which methionine residue at position 256 is replaced with a glutamic acid residue (E. coli JM39-8(pCEM256E), private number: AJ13391) was deposited at the National Institute of Bioscience and Human-Technology, Agency of Industrial Science and Technology (Postal code: 305, 1-3 Higashi 1-Chome, Tsukuba-shi, Ibaraki-ken, Japan) on Nov. 20, 1997 and assigned an accession number of FERM P-16527. The deposit was then converted to an international deposit under the provisions of Budapest Treaty on Jul. 8, 2002, and assigned an accession number of FERM BP-8112.

Although a “SAT insensitive to feedback inhibition by L-cysteine” can be SAT which has been modified so that it is insensitive to the feedback inhibition by L-cysteine, it can be a SAT which in its native form is insensitive to the feedback inhibition by L-cysteine. For example, SAT of Arabidopsis thaliana is known to be not subject to the feedback inhibition by L-cysteine and can be suitably used. As a plasmid containing the SAT gene derived from Arabidopsis thaliana, pEAS-m is known (FEMS Microbiol. Lett., 179 (1999) 453-459).

Furthermore, the ability to produce L-cysteine can also be improved by enhancing expression of the cysPTWAM cluster genes coding for the sulfate/thio sulfate transport system proteins (Japanese Patent Laid-open No. 2005-137369, EP 1528108).

Furthermore, a sulfide can be incorporated into O-acetyl-L-serine via a reaction catalyzed by the O-acetylserine (thiol)-lyase A or B encoded by the cysK and cysM genes, respectively, to produce L-cysteine. Therefore, the ability to produce L-cysteine can also be improved by enhancing expression of the genes coding for these enzymes.

Moreover, L-cysteine-producing ability can also be improved by suppressing the L-cysteine decomposition system. The phrase “L-cysteine decomposition system is suppressed” can mean that intracellular L-cysteine decomposition activity is decreased as compared to that of a non-modified strain such as a wild-type or parent strain. As proteins responsible for the L-cysteine decomposition system, cystathionine-β-lyase (metC product, Japanese Patent Laid-open No. 11-155571, Chandra et al., Biochemistry, 21 (1982) 3064-3069), tryptophanase (tnaA product, Japanese Patent Laid-open No. 2003-169668, Austin Newton et al., J. Biol. Chem., 240 (1965) 1211-1218)), O-acetylserine sulfhydrylase B (cysM gene product, Japanese Patent Laid-open No. 2005-245311) and the malY gene product (Japanese Patent Laid-open No. 2005-245311) are known. By decreasing the activities of these proteins, L-cysteine-producing ability can be improved.

Modifications for decreasing the activity of a protein can be attained in the same manner as those for the fliY or ydjN gene described later.

The nucleotide sequence of the cysM gene of Escherichia coli and the amino acid sequence encoded by the gene are shown in SEQ ID NOS: 25 and 26, respectively.

Decrease of Activities of YdjN Protein and FliY Protein

The bacterium in accordance with the presently disclosed subject matter can be obtained by modifying a bacterium belonging to the family Enterobacteriaceae with L-cysteine-producing ability as described above to decrease the activity of the YdjN protein, or the activities of the YdjN protein and the FliY protein. After a bacterium is modified to decrease the activity of the YdjN protein or the activities of the YdjN and FliY proteins, L-cysteine-producing ability may be imparted to the bacterium. The YdjN protein and the FliY protein are proteins encoded by the ydjN gene and the fliY gene, respectively. The activities of the YdjN and FliY proteins of a bacterium can be decreased by, for example, modifying the bacterium having the fliY and ydjN genes to decrease the activities of FliY and YdjN encoded by these genes. In order to enhance the L-cysteine-producing ability, either the FliY activity or the YdjN activity may be decreased, but only the YdjN activity can be decreased, and both the activities can be decreased in another example.

The “decrease” of activity can include decrease of the activity of a modified strain to a level lower than that of a wild-type or a non-modified strain, and complete disappearance of the activity, unless otherwise specified.

Novel genes coding for proteins of which deletion from the chromosomal DNA of Pantoea ananatis enhanced the L-cysteine-producing ability, and designated them fliY and ydjN, respectively, since they showed high homology to fliY and ydjN of E. coli (78% and 80%, respectively). In this specification, “homology” may means “identity”.

In the present invention, in addition to the fliY and ydjN genes of E. coli, fliY and ydjN genes of Pantoea ananatis, and homologue genes of those genes of other bacteria may also be called fliY gene and ydjN gene, respectively.

Specific examples of the fliY gene include a gene comprising the nucleotide sequence shown in SEQ ID NO: 5 or 7. Specific examples of the ydjN gene include a gene comprising the nucleotide sequence shown in SEQ ID NO: 1 or 3.

The fliY gene of the Escherichia coli MG1655 strain is shown in SEQ ID NO: 5, and the amino acid sequence encoded by the gene is shown in SEQ ID NO: 6. The fliY gene of the Pantoea ananatis SC17 strain is shown in SEQ ID NO: 7, and the amino acid sequence encoded by the gene is shown in SEQ ID NO: 8.

The nucleotide sequence of the ydjN gene of the Escherichia coli MG1655 strain is shown in SEQ ID NO: 1, and the amino acid sequence encoded by the gene is shown in SEQ ID NO: 2. The nucleotide sequence of the ydjN gene of the Pantoea ananatis SC17 strain is shown in SEQ ID NO: 3, and the amino acid sequence encoded by the gene is shown in SEQ ID NO: 4.

The FliY and YdjN proteins are not limited to proteins having the aforementioned amino acid sequences and homologues thereof, and they may be a variant thereof. The fliY or ydjN gene may be a gene coding for a variant of the FliY or YdjN protein. A variant of the FliY or the YdjN protein means a protein having the amino acid sequence of SEQ ID NO: 2, 4, 6 or 8 including substitutions, deletions, insertions or additions of one or several amino acid residues at one or several positions, and having the function of the FliY or YdjN protein. Although the number meant by the aforementioned term “one or several” may differ depending on positions of amino acid residues in the three-dimensional structure of the protein or the types of amino acid residues, specifically, it can be 1 to 20, 1 to 10, or even 1 to 5.

A ydjN gene-deficient strain shows decreased uptake of S-sulfocysteine and L-cystine as shown in Examples section. Therefore, it is estimated that the YdjN protein has a function to participate in uptake of S-sulfocysteine and L-cystine.

On the other hand, although there is a reference pointing out the possibility of participation of FliY in uptake of L-cystine (Butler et al., Life Sci., 52, 1209-1215 (1993); Hosie et al., Res. Microbiol., 152, 259-270 (2001)), it is estimated that it does not participate in the uptake of L-cystine, or the activity thereof is lower than that of YdjN, if it participates in that uptake, as shown in Examples section. In any case, if both the ydjN and fliY genes are deleted, the L-cysteine-producing ability is markedly increased as compared with that obtainable by deletion of either one of them. Therefore, although the function of FliY is still indefinite, it is characterized that deletion thereof improves the L-cysteine-producing ability.

The aforementioned substitutions, deletions, insertions, or additions of one or several amino acid residues are a conservative mutation that preserves the normal function of the protein.

The conservative mutation is typically a conservative substitution. The conservative substitution is a mutation wherein substitution takes place mutually among Phe, Trp and Tyr, if the substitution site is an aromatic amino acid; among Leu, Ile and Val, if the substitution site is a hydrophobic amino acid; between Gln and Asn, if it is a polar amino acid; among Lys, Arg and His, if it is a basic amino acid; between Asp and Glu, if it is an acidic amino acid; and between Ser and Thr, if it is an amino acid having a hydroxyl group. Specific examples of conservative substitutions include: substitution of Ser or Thr for Ala; substitution of Gln, His or Lys for Arg; substitution of Glu, Gln, Lys, His or Asp for Asn; substitution of Asn, Glu or Gln for Asp; substitution of Ser or Ala for Cys; substitution of Asn, Glu, Lys, His, Asp or Arg for Gln; substitution of Gly, Asn, Gln, Lys or Asp for Glu; substitution of Pro for Gly; substitution of Asn, Lys, Gln, Arg or Tyr for His; substitution of Leu, Met, Val or Phe for Ile; substitution of Ile, Met, Val or Phe for Leu; substitution of Asn, Glu, Gln, His or Arg for Lys; substitution of Ile, Leu, Val or Phe for Met; substitution of Trp, Tyr, Met, Ile or Leu for Phe; substitution of Thr or Ala for Ser; substitution of Ser or Ala for Thr; substitution of Phe or Tyr for Trp; substitution of His, Phe or Trp for Tyr; and substitution of Met, Ile or Leu for Val. The above-mentioned amino acid substitution, deletion, insertion, addition, inversion etc. can be the result of a naturally-occurring mutation (mutant or variant) due to an individual difference, a difference of species, or the like of a bacterium from which the gene is derived.

Furthermore, the gene having such a conservative mutation as described above can be a gene encoding a protein showing a homology of 80% or more, 90% or more, 95% or more, 97% or more, or even 99% or more, to the entire encoded amino acid sequence. Sequence information of the genes coding for a protein homologous to such FliY or YdjN can be easily obtained from databases opened to public by BLAST searching or FASTA searching using the wild-type fliY or ydjN gene of the aforementioned Escherichia coli strain as a query sequence, and the genes can be obtained by using oligonucleotides produced based on such known gene sequences as primers.

The fliY or YdjN gene can be a gene which hybridizes with a sequence complementary to the aforementioned nucleotide sequences or a probe that can be prepared from the aforementioned nucleotide sequences under stringent conditions, so long as the function of the protein encoded by the fliY or YdjN gene is maintained. Examples of the “stringent conditions” include conditions of washing at 60° C., 1×SSC, 0.1% SDS, 60° C., 0.1×SSC, 0.1% SDS in another example, once or twice or three times in another example.

The probe used for the aforementioned hybridization can have a partial sequence of a complementary sequence of the gene. Such a probe can be prepared by PCR using oligonucleotides prepared based on the known nucleotide sequences of the gene as primers, and a DNA fragment containing these sequences as the template. When a DNA fragment of a length of about 300 by is used as the probe, the conditions of washing after hybridization can be, for example, 50° C., 2×SSC, and 0.1% SDS.

Methods for decreasing the activities of the FliY or YdjN protein will be explained below. The activities of the proteins of the L-cysteine decomposition system can also be decreased by the same methods. In the following descriptions, an objective protein of which activity is to be decreased is referred to as a “target protein”, and a gene coding for the target protein is referred to as a “target gene”.

Activity of a target protein can be decreased by, for example, reducing expression of a target gene. Specifically, for example, intracellular activity of the target protein can be reduced by deleting a part of, or the entire coding region of the target gene on a chromosome. For decrease of activity of a target protein, expression of the target gene can also be decreased by modifying an expression control sequence of the target gene such as promoter and Shine-Dalgarno (SD) sequence. Furthermore, the expression amount of the gene can also be reduced by modification of a non-translation region other than the expression control sequence. Furthermore, the entire gene including the sequences on both sides of the gene on a chromosome can be deleted. Furthermore, the expression of the gene can also be reduced by introducing a mutation for an amino acid substitution (missense mutation), a stop codon (nonsense mutation), or a frame shift mutation which adds or deletes one or two nucleotides into the coding region of the target gene on a chromosome (Journal of Biological Chemistry, 272:8611-8617 (1997); Proceedings of the National Academy of Sciences, USA, 95 5511-5515 (1998); Journal of Biological Chemistry, 266, 20833-20839 (1991)). Activity of a target protein can also be decreased by enhancing activity of a regulator which down-regulates the target protein, or suppressing activity of a regulator which up-regulates the target protein. Activity of a target protein can also be decreased by adding a substance which down-regulates activity or expression of the target protein, or eliminating a substance which up-regulates activity or expression of the target protein.

Furthermore, the modification can be a modification caused by a typical mutagenesis caused by X-ray or ultraviolet irradiation, or by use of a mutagen such as N-methyl-N′-nitro-N-nitrosoguanidine, so long as the modification results in a decrease of the activity of the target protein.

Modification of an expression control sequence is performed for one or more nucleotides in one example, two or more nucleotides, three or more nucleotides in another example. When a coding region is deleted, the region to be deleted can be an N-terminal region, an internal region or a C-terminal region, or even the entire coding region, so long as the function of the target protein is decreased or deleted. Deletion of a longer region can usually more surely inactivate a gene. Furthermore, reading frames upstream and downstream of the region to be deleted can be the same or different.

To inactivate a gene by inserting a sequence into the coding region of the gene, the sequence can be inserted into any part of the coding region of the gene. The longer the inserted sequence, the greater the likelihood of inactivating the gene. Reading frames located upstream and downstream of the insertion site can be the same or different. The sequence to be inserted is not particularly limited so long as the insertion decreases or deletes the function of the encoded target protein, and examples include, for example, a transposon carrying an antibiotic resistance gene, a gene useful for L-cysteine production and so forth.

A target gene on the chromosome can be modified as described above by, for example, preparing a deletion-type version of the gene in which a partial sequence of the gene is deleted so that the deletion-type version of the gene does not produce a target protein which normally functions, and transforming a bacterium with a DNA containing the deletion-type gene to cause homologous recombination between the deletion-type gene and the native gene on the chromosome, and thereby substitute the deletion-type gene for the gene on the genome. The target protein encoded by the deletion-type gene has a conformation different from that of the wild-type protein, if it is even produced, and thus the function is reduced or deleted. Such gene disruption based on gene substitution utilizing homologous recombination has been already established, and there are a method called Red-driven integration (Datsenko, K. A., and Wanner, B. L., Proc. Natl. Acad. Sci. USA, 97:6640-6645 (2000)), a method of using a linear DNA such as a method utilizing the Red driven integration in combination with an excision system derived from phage (Cho, E. H., Gumport, R. I., Gardner, J. F., J. Bacteriol., 184:5200-5203 (2002)) (refer to WO2005/010175), a method of using a plasmid containing a temperature sensitive replication origin or a plasmid capable of conjugative transfer, a method of utilizing a suicide vector not having replication origin in a host (U.S. Pat. No. 6,303,383, Japanese Patent Laid-open No. 05-007491), and so forth.

Decrease of transcription level of a target gene can be confirmed by comparing amount of mRNA transcribed from the gene with that of the wild-type or non-modified strain. Examples of the method for confirming mRNA amount include Northern hybridization, RT-PCR (Molecular Cloning, Cold Spring Harbor Laboratory Press, Cold Spring Harbor (USA), 2001), and so forth.

Decrease of amount of a target protein can also be confirmed by Western blotting using an antibody (Molecular Cloning, Cold Spring Harbor Laboratory Press, Cold Spring Harbor (USA), 2001).

Furthermore, when the target protein is the YdjN protein, decrease of the amount of the protein can also be confirmed by measuring activity to take up S-sulfocysteine or L-cystine of the cell.

Whether a protein has the activity to take up the aforementioned compound can be confirmed by preparing a bacterium in which expression of a gene coding for the protein is increased from a wild strain or a parent strain, culturing this strain in a medium, and quantifying amount of L-cysteine, L-cystine, a derivative or precursor thereof, or a mixture of them accumulated in the medium. Alternatively, the activity can also be confirmed by preparing a bacterium in which expression of a gene coding for the protein is decreased or deleted from a wild-type or parent strain, culturing the strain in a medium containing S-sulfocysteine or L-cystine, and confirming decrease of the decreased amount of the compound added to the medium. Specific examples are described in Examples section.

When the fliY or ydjN gene of Escherichia coli is used as the fliY or ydjN gene, the fliY or ydjN gene can be obtained by PCR using chromosomal DNA of Escherichia coli as a template and primers prepared on the basis of the nucleotide sequence of SEQ ID NO: 5 or 1. Similarly, the fliY or ydjN gene of Pantoea ananatis can be obtained by PCR using chromosomal DNA of Pantoea ananatis as a template and primers prepared on the basis of the nucleotide sequence of SEQ ID NO: 7 or 3. The fliY or ydjN gene of other bacteria can also be obtained from chromosomes or chromosomal DNA library of the bacteria by hybridization or PCR using a probe or primers prepared on the basis of the aforementioned sequence information.

When it is necessary to increase expression of the fliY or ydjN gene in order to confirm whether the FliY or YdjN protein has the activity to take up S-sulfocysteine, L-cystine or L-cysteine, multiple copies of the gene can be introduced into a bacterium. In order to introduce multiple copies of the fliY or ydjN gene into a bacterium, the method of using a multi-copy type vector, the method of introducing multiple copies of genes into chromosomal DNA by homologous recombination, and so forth can be used as described for the SAT gene.

### <2> Method for Producing L-Cysteine, L-Cystine, Derivative or Precursor Thereof or Mixture Thereof

These compounds can be produced by culturing the bacterium in accordance with the presently disclosed subject matter obtained as described above in a medium, and collecting L-cysteine, L-cystine, a derivative or precursor thereof or a mixture thereof from the medium. Examples of the derivative or precursor of L-cysteine include S-sulfocysteine, a thiazolidine derivative, a hemithioketal corresponding the thiazolidine derivative mentioned above, and so forth.

Examples of the medium used for the culture can include ordinary media containing a carbon source, nitrogen source, sulfur source, inorganic ions, and other organic components as required.

As the carbon source, saccharides such as glucose, fructose, sucrose, molasses and starch hydrolysate, and organic acids such as fumaric acid, citric acid and succinic acid can be used.

As the nitrogen source, inorganic ammonium salts such as ammonium sulfate, ammonium chloride and ammonium phosphate, organic nitrogen such as soybean hydrolysate, ammonia gas, aqueous ammonia and so forth can be used.

As the sulfur source, inorganic sulfur compounds, such as sulfates, sulfites, sulfides, hyposulfites and thiosulfates can be used.

As organic trace amount nutrients, it is desirable to add required substances such as vitamin B₁, yeast extract and so forth in appropriate amounts. Other than these, potassium phosphate, magnesium sulfate, iron ions, manganese ions and so forth are added in small amounts.

The culture can be performed under aerobic conditions for 30 to 90 hours. The culture temperature can be controlled to be at 25° C. to 37° C., and pH can be controlled to be 5 to 8 during the culture. For pH adjustment, inorganic or organic acidic or alkaline substances, ammonia gas and so forth can be used. Collection of L-cysteine from the culture can be attained by, for example, any combination of known ion exchange resin methods, precipitation and other known methods.

L-Cysteine obtained as described above can be used for production of L-cysteine derivatives. The cysteine derivatives include methylcysteine, ethylcysteine, carbocysteine, sulfocysteine, acetylcysteine, and so forth.

Furthermore, when a thiazolidine derivative of L-cysteine is accumulated in the medium, L-cysteine can be produced by collecting the thiazolidine derivative from the medium to break the reaction equilibrium between the thiazolidine derivative and L-cysteine so that L-cysteine is excessively produced. Furthermore, when S-sulfocysteine is accumulated in the medium, it can be converted into L-cysteine by reduction with a reducing agent such as dithiothreitol.

L-Cysteine, a derivative thereof, and so forth collected in the present invention may contain cells of microorganism, medium components, moisture, and by-products of microbial metabolism in addition to the objective compound. Purity of the collected objective compound is 50% or higher in one example, 85% or higher, 95% or higher in another example.

### EXAMPLES

Hereinafter, the present invention will be explained more specifically with reference to the following non-limiting examples.

In the following descriptions, cysteine can mean L-cysteine.

### Example 1

### Identification of a Protein which has an Activity of Taking Up Cysteine or Cystine

(1) Acquisition of Mutant Strain Unable to Utilize S-Sulfocysteine as a Sole Cysteine Source

(1-1) Acquisition of a Strain from E. coli MG1655 Strain (ATCC No. 47076) which Lacks the cysE Gene.

The cysE gene was deleted by the method called “Red-driven integration” developed by Datsenko, Wanner et al. (Proc. Natl. Acad. Sci. USA, 2000, vol. 97, No. 12, pp. 6640-6645) and the excisive system derived from λ phage (J. Bacteriol., 2000, 184, 5200-5203 (2002)). According to the Red-driven integration, a gene-disrupted strain can be constructed in one step by using a PCR product obtained with synthetic oligonucleotides designed so as to have a part of the objective gene on the 5′ side, and a part of an antibiotic resistance gene on the 3′ side. By further combining the excisive system derived from λ-phage, the antibiotic resistance gene incorporated into the gene-disrupted strain can be eliminated. Methods for deleting a gene of E. coli using this Red-driven integration and the excisive system derived from λ-phage are described in detail in Japanese Patent Laid-open No. 2005-058227, WO2007/119880, and so forth. A cysE gene-deficient strain was also obtained in the same manner.

A DNA fragment containing an antibiotic resistance gene (kanamycin resistance gene (Km^{r})) between sequences homologous to the both ends of the cysE gene was obtained by PCR. Specific experimental procedure and experimental materials were the same as those described in Japanese Patent Laid-open No. 2005-058227, except that DcysE(Ec)-F (ccggcccgcg cagaacgggc cggtcattat ctcatcgtgt ggagtaagca tgaagcctgc ttttttatac taagttggca, SEQ ID NO: 50), and DcysE(Ec)-R (actgtaggcc ggatagatga ttacatcgca tccggcacga tcacaggaca cgctcaagtt agtataaaaa agctgaacga, SEQ ID NO: 51) were used as primers, and pMW118-(λattL-Km-λattR) (WO2006/093322A2) was used as the template. The obtained deficient strain was designated MG1655ΔcysE.

(1-2) Preparation of Transposon-Mutated Strain Library from the MG1655ΔcysE Strain

By using EZ-Tn5<KAN-2> Tnp Transposome Kit (EPICENTRE), a library of mutant strains in which Tn5 was randomly inserted was prepared from the MG1655ΔcysE strain. As for specific experimental procedure, the experiment was performed according to the kit instructions.

(1-3) Screening of Mutant Strain Library for Mutant Strain that is Unable to Utilize S-Sulfocysteine as the Sole Cysteine Source

The aforementioned library was screened for a mutant strain that is unable to utilize S-sulfocysteine as the sole cysteine source. The “cysteine source” can refer to a substrate that is taken up into cells and used for the production of cysteine. When cysteine cannot be synthesized in cells, the cysteine source can be cysteine itself. The mutant strains were each spotted with a toothpick on either M9 agar medium (Sambrook and Russell, Molecular Cloning: A Laboratory Manual (Third Edition), Cold Spring Harbor Laboratory Press) containing 20 μM cysteine, or M9 agar medium containing 20 μM S-sulfocysteine (cat #C2196, SIGMA), to screen for a mutant strain able to grow on cysteine-containing medium, but unable to grow on an S-sulfocysteine-containing medium. One mutant strain from about 1000 strains was obtained. When the genome region of the inserted Tn5 was identified, it was found that Tn5 had been inserted into the ydjN gene.

(1-4) Analysis of S-Sulfocysteine-Assimilating Ability of a Strain Lacking the ydjN Gene

In order to elucidate whether the phenotype of the mutant strain obtained by insertion of Tn5 was due to functional deficiency of the ydjN gene, a strain lacking the ydjN gene was constructed from the MG1655ΔcysE strain using Red-driven integration, described above. For this construction, primers DydjN(Ec)-F (cactatgact gctacgcagt gatagaaata ataagatcag gagaacgggg tgaagcctgc ttttttatac taagttggca, SEQ ID NO: 52), and DydjN(Ec)-R (aaagtaaggc aacggcccct atacaaaacg gaccgttgcc agcataagaa cgctcaagtt agtataaaaa agctgaacga, SEQ ID NO: 53) were used. The constructed deficient strain was designated MG1655ΔcysEΔydjN::Km strain. MG1655ΔydjN::Km strain was also obtained from MG1655 by the same method.

Growth of the strains on the M9 agar medium (provided that MgCl₂ was used instead of MgSO₄ at the same concentration as the medium component) containing 50 μM cysteine, 50 μM cystine or 50 μM S-sulfocysteine is shown in Table 1.

TABLE 1

| | M9 | M9 + | M9 + S- | |
|---|---|---|---|---|
| Strain | (w/o sulfur) | cysteine | sulfocysteine | M9 + cystine |
| MG1655 | + | + | + | + |
| MG1655ΔcysE | − | + | + | + |
| MG1655ΔydjN::Km | + | + | + | + |

Although no source of sulfur was added to the M9 agar medium in this experiment (M9 w/o sulfur), the cysE-non-disrupted strains (MG1655 strain, MG1655ΔydjN::Km strain etc.) are able to grow with just a trace amount of contaminating sulfur compounds. Therefore, to investigate whether S-sulfocysteine can be used as a cysteine sourceor not, the background of the cysE deficiency should be determined. That is, the MG1655ΔcysE strain cannot grow without a cysteine source (w/o sulfur), but can grow by using cysteine, cystine or S-sulfocysteine as a cysteine source, if they are present. It was found that the ydjN-deficient strain can grow with cysteine or cystine, but not S-sulfocysteine, as a cysteine source (Table 1, MG1655ΔcysEΔydjN::Km strain). From this result, it was found that the ydjN gene is indispensable for the assimilation of S-sulfocysteine. Alternatively, even if ydjN was deleted, cysteine and cystine can be assimilated.

(2) Functional Analysis of ydjN and fliY

(2-1) Cloning of ydjN gene from E. coli MG1655 strain and P. ananatis SC17 strain

When the ydjN gene was cloned from the E. coli MG1655 strain and the Pantoea ananatis SC17 strain (U.S. Pat. No. 6,596,517), expression vectors based on pMIV-Pnlp8 and pMIV-Pnlp0 were used. The potent nlp8 promoter (or nip0 promoter) and an rrnB terminator were integrated into these expression vectors, and by inserting a target gene between the promoter and the terminator; it functions as an expression unit. “Pnlp0” indicates a promoter of the wild-type nlpD gene, and “Pnlp8” indicates a mutant promoter of the nlpD gene. The details of the construction of these expression vectors are described in Example 3 as construction of pMIV-Pnlp8-YeaS7 and pMIV-Pnlp0-YeaS3. In pMIV-Pnlp8-yeaS7 and pMIV-Pnlp0-yeaS3, the yeaS gene is cloned between the nlp8 or nip0 promoter and the rrnB terminator using the SalI and XbaI sites. If the SalI and XbaI sites are designed in primers beforehand, the ydjN gene can also be inserted into those vectors in the same manner as that for yeaS. That is, the expression plasmids to be constructed correspond to expression plasmids having structures of pMIV-Pnlp8-yeaS7 and pMIV-Pnlp0-yeaS3 mentioned later in which the yeaS gene is replaced by the ydjN gene.

The ydjN gene of E. coli was amplified by using the genomic DNA of the MG1655 strain as a template, as well as ydjN(Ec)-SalIFW2 (acgcgtcgac atgaactttc cattaattgc gaacatcgtg gtg, SEQ ID NO: 54) and ydjN(Ec)-xbaIRV2 (ctagtctaga ttaatggtgt gccagttcgg cgtcg, SEQ ID NO: 55) as primers, with a PCR cycle of 94° C. for 5 minutes, followed by 30 cycles of 98° C. for 5 seconds, 55° C. for 5 seconds and 72° C. for 90 seconds, and final incubation at 4° C. In the case of P. ananatis, the ydjN gene was amplified by using the genomic DNA of the SC17 strain as a template, as well as ydjN2(Pa)-SalIFW (acgcgtcgac atggatattc ctcttacgc, SEQ ID NO: 56) and ydjN2(Pa)-xbaIRV (tgctctagat tagctgtgct ctaattcac, SEQ ID NO: 57) as primers, with a PCR cycle of 94° C. for 5 minutes, followed by 30 cycles of 98° C. for 5 seconds, 55° C. for 5 seconds and 72° C. for 2 minutes, and final incubation at 4° C. SalI and XbaI sites were designed at the both ends in all the primers. The amplified fragments were each integrated into the pMIV-Pnlp0 vector, and the constructed plasmids were designated according to the origin of the gene (E. coli (Ec) or P. ananatis (Pa)) as pMIV-Pnlp0-ydjN(Ec) and pMIV-Pnlp0-ydjN(Pa), respectively. pMIV-5JS (Japanese Patent Laid-open No. 2008-99668) was used as a control.

(2-2) Functional Analysis of ydjN

When ydjN was deleted, the strains could not grow with S-sulfocysteine as the sole cysteine source. Therefore, it was suspected that the ydjN gene might code for a transporter (uptake factor) of S-sulfocysteine. Therefore, it was examined whether there was any difference in the ability to take up S-sulfocysteine between the ydjN-deficient strain of the MG1655 strain (MG1655ΔydjN::Km strain described above) and ydjN-enhanced strain of the MG1655 strain (MG1655 strain transformed with pMIV-Pnlp0-ydjN(Ec)). A similar investigation was also conducted for cystine and cysteine, which are compounds similar to S-sulfocysteine.

The S-sulfocysteine uptake experiment was performed as follows. First, the MG1655ΔydjN::Km strain and a control strain, MG1655 strain, as well as the MG1655/pMIV-Pnlp0-ydjN(Ec) strain and a control strain, MG1655/pMIV-5JS strain, were cultured overnight in LB liquid medium (3-ml test tube, 37° C., shaking culture). The cells were collected from the culture medium, washed twice with the M9 minimal medium containing 0.4% glucose, and then suspended in the M9 minimal medium containing 0.4% glucose at a density two times that of the original culture medium. The cell suspension of each strain prepared as described above was inoculated into a volume of 40 μl to 4 ml of the M9 minimal medium containing 0.4% glucose, and culture was performed at 37° C. with shaking by using an automatically OD measuring culture apparatus, BIO-PHOTORECORDER TN-1506 (ADVANTEC).

When the OD reached around 0.3 (culture for about 5 hours), 20 μl of 100 mM S-sulfocysteine was added (final concentration: 0.5 mM), and the medium was sampled (0.2 ml of the culture medium was taken and mixed with 0.8 ml of 1 N hydrochloric acid) over time for 2 hours after the addition of S-sulfocysteine (0 hour). Amino acid analysis of the sample at each time point was performed with an amino acid analyzer (L-8900, Hitachi), and S-sulfocysteine concentration in the medium was determined by comparison with a 0.4 mM standard sample similarly prepared with 1 N hydrochloric acid. In the culture, 25 mg/L of chloramphenicol was added to the medium for all the plasmid-harboring strains.

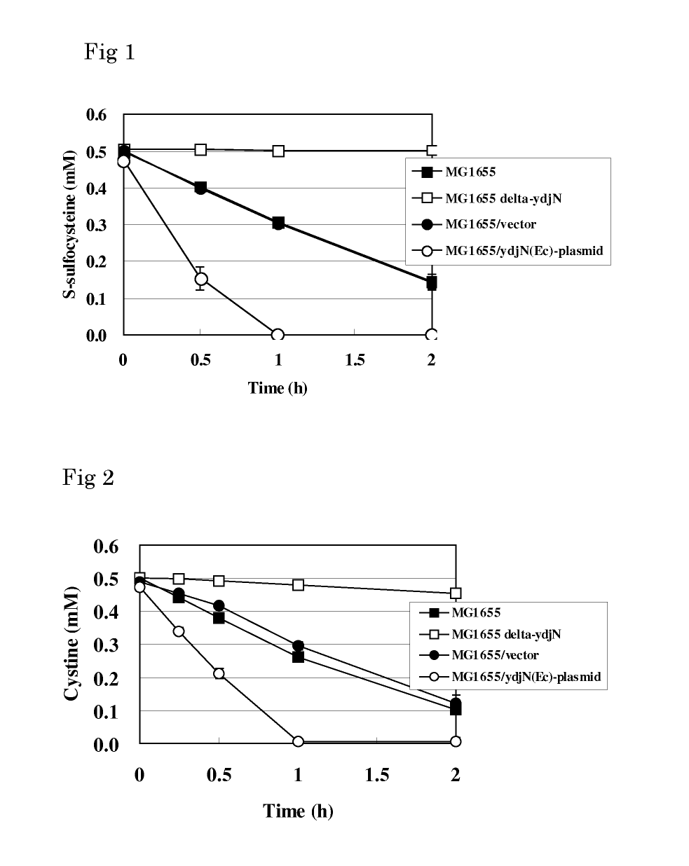

The change in S-sulfocysteine concentration in the culture medium for each strain is shown in FIG. 1. In the graph, the MG1655/pMIV-Pnlp0-ydjN(Ec), MG1655/pMIV-5JS, and MG1655ΔydjN::Km strains are abbreviated as MG1655/ydjN(Ec)-plasmid, MG1655/vector, and MG1655 delta-ydjN, respectively. It was found that the S-sulfocysteine concentration in the medium gradually decreased with the wild-type strain, but S-sulfocysteine did not decrease at all with the ydjN-deficient strain, and decrease of S-sulfocysteine was accelerated with the ydjN-enhanced strain, as compared with the control strains. Furthermore, when YdjN was analyzed with a membrane protein prediction program SOSUI (http://bp.nuap.nagoya-u.ac.jp/sosui/), ten transmembrane domains were found, and therefore it was expected to be a membrane protein. The results described above strongly suggested the possibility that ydjN coded for a transporter (uptake factor) of S-sulfocysteine. Furthermore, since when ydjN is deficient, S-sulfocysteine was unable to be assimilated (Table 1), ydjN is considered to be the sole S-sulfocysteine transporter in E. coli.

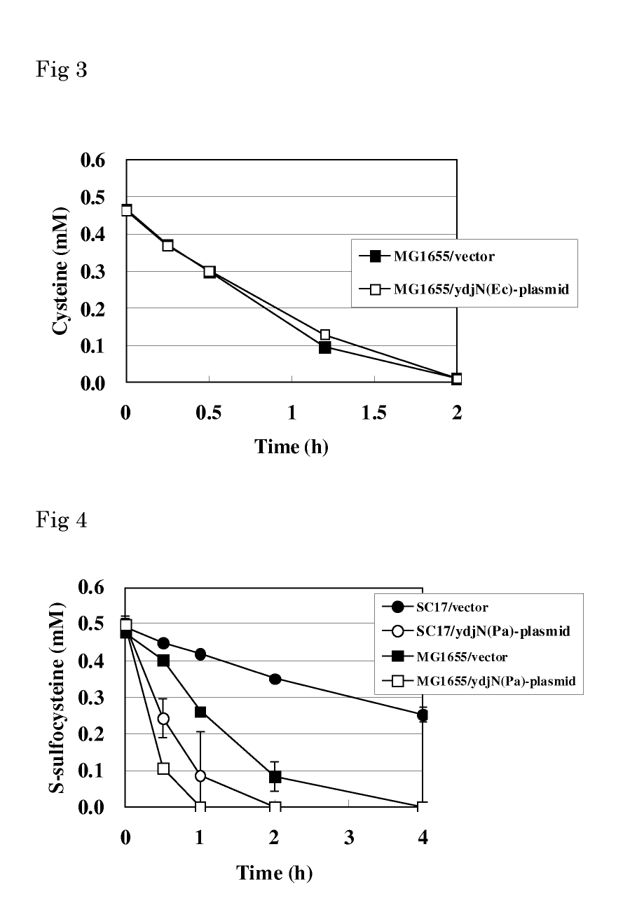

Furthermore, uptake of cystine or cysteine by YdjN was also examined by using the same experimental system, and adding cystine or cysteine as a substrate instead of S-sulfocysteine. The results are shown in FIGS. 2 and 3. The strain names in the graphs are the same as those used in FIG. 1.

As shown in FIG. 2, the same results as those for S-sulfocysteine were obtained when cystine was used as the substrate, and therefore it was suggested that YdjN had the activity to take up cystine. In the ydjN-deficient strain, uptake of cystine markedly decreased, and the residual activity to take up cystine became extremely weak. Therefore, it is considered that YdjN is a major transporter of cystine in the E. coli MG1655 strain. However, since the ydjN-deficient strain could also grow with cystine as the cysteine source (Table 1), it was considered that there is likely another active transporter of cysteine, other than YdjN. Alternatively, when cysteine was used as the substrate, uptake was not increased even when ydjN was enhanced, as shown in FIG. 3, and therefore it was suspected that it might not participate in the uptake of cysteine.

Furthermore, the plasmid pMIV-Pnlp0-ydjN(Pa) expressing ydjN derived from P. ananatis was introduced into E. coli and P. ananatis to enhance ydjN, and uptake of S-sulfocysteine was examined. The results are shown in FIG. 4. In the graph, pMIV-Pnlp0-ydjN(Pa) and pMIV-5JS are abbreviated as ydjN(Pa)-plasmid and vector, respectively.

It was confirmed that YdjN of P. ananatis also had the activity to take up S-sulfocysteine, like YdjN of E. coli. Furthermore, the amino acid sequences of YdjN of P. ananatis and YdjN of E. coli show a homology of 80%.

(2-3) Functional Analysis of fliY

Then, in order to examine whether fliY participates in uptake of cystine and cysteine, fliY-deficient and enhanced strains were constructed.

The fliY gene was deleted by using the aforementioned Red-driven integration and excisive system derived from λ-phage. DfliY(Ec)-FW (atgaaattag cacatctggg acgtcaggca ttgatgggtg tgatggccgt tgaagcctgc ttttttatac taagttggca, SEQ ID NO: 58) and DfliY(Ec)-RV (ttatttggtc acatcagcac caaaccattt ttcggaaagg gcttgcagag cgctcaagtt agtataaaa agctgaacga, SEQ ID NO: 59) were used as primers, and pMW118-(λattL-Cm^{R}-λattR) (Katashkina ZhI et al., Mol. Biol. (Mosk), 39(5):823-31, 2005) was used as the template. MG1655ΔfliY strain was obtained from the MG1655 strain, and MG1655ΔydjN::KmΔfliY::Cm strain was obtained from the MG1655ΔydjN::Km strain described above.

Furthermore, in order to enhance the fliY gene using a plasmid, pMIV-Pnlp0-fliY(Ec) was constructed. The construction method was the same as the method of cloning the ydjN gene into pMIV-Pnlp0 mentioned above, but in this experiment, for amplification of the fliY gene, fliY(Ec)SalI-F (acgcgtcgac atgaaattag cacatctggg acg, SEQ ID NO: 60) and fliY(Ec)XbaI-R (ctagtctaga ttatttggtc acatcagcac c, SEQ ID NO: 61) were used as primers.

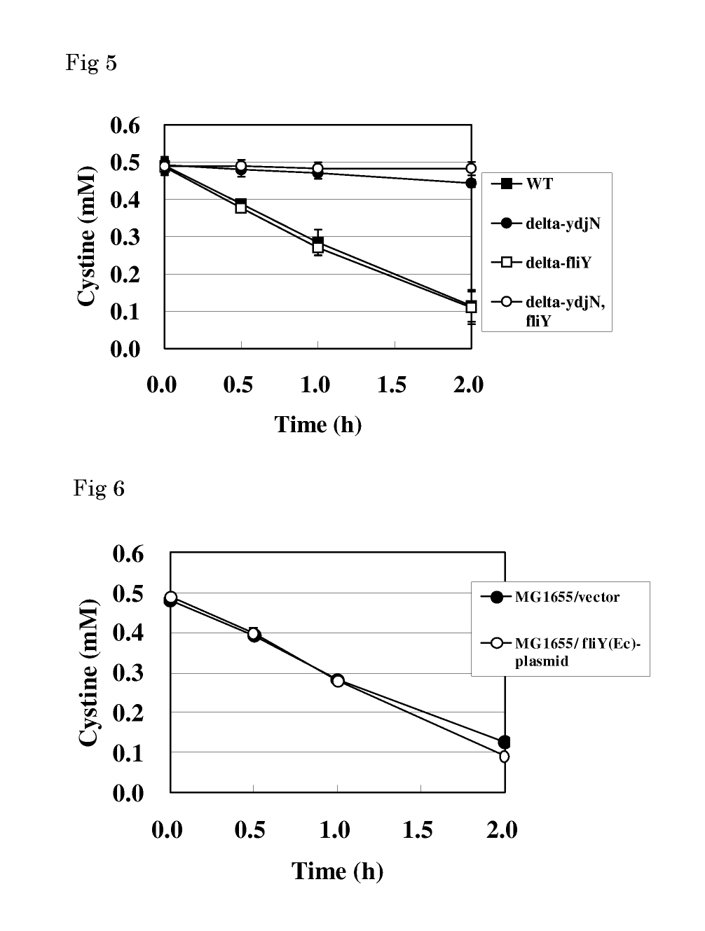

First, in order to investigate the effect of fliY deficiency on cystine uptake, an uptake experiment was performed by using cystine as a substrate with 4 strains, the MG1655, MG1655ΔydjN::Km, MG1655ΔfliY, and MG1655ΔydjN::KmΔfliY::Cm. The results are shown in FIG. 5. In the graph, “WT” represents the MG1655 strain, “delta-ydjN” represents the MG1655ΔydjN::Km strain, “delta fliY” represents the MG1655ΔfliY strain, and “delta-ydjN,fliY” represents the MG1655ΔydjN::KmΔfliY::Cm strain. As a result, the cystine uptake rate of the MG1655ΔfliY strain, in which only the fliY gene was deleted, was not different from that of the MG1655 strain (FIG. 5). Furthermore, although it was observed that the cystine uptake rate of the MG1655ΔydjNΔfliY strain, which was a fliY and ydjN gene-double deficient strain, seemed to decrease as compared with that of the MG1655ΔydjN strain, in which only ydjN was deficient, the difference was very small, and therefore it was unclear whether a significant difference was induced by the fliY deficiency (FIG. 5).

Furthermore, uptake of cystine by the fliY-enhanced E. coli MG1655 strain was also investigated. The results are shown in FIG. 6. In the graph, “fliY(Ec)-plasmid” represents pMIV-Pnlp0-fliY(Ec), and “vector” represents pMIV-5JS. Also when fliY was enhanced, a significant difference in the uptake of cystine was not observed (FIG. 6).

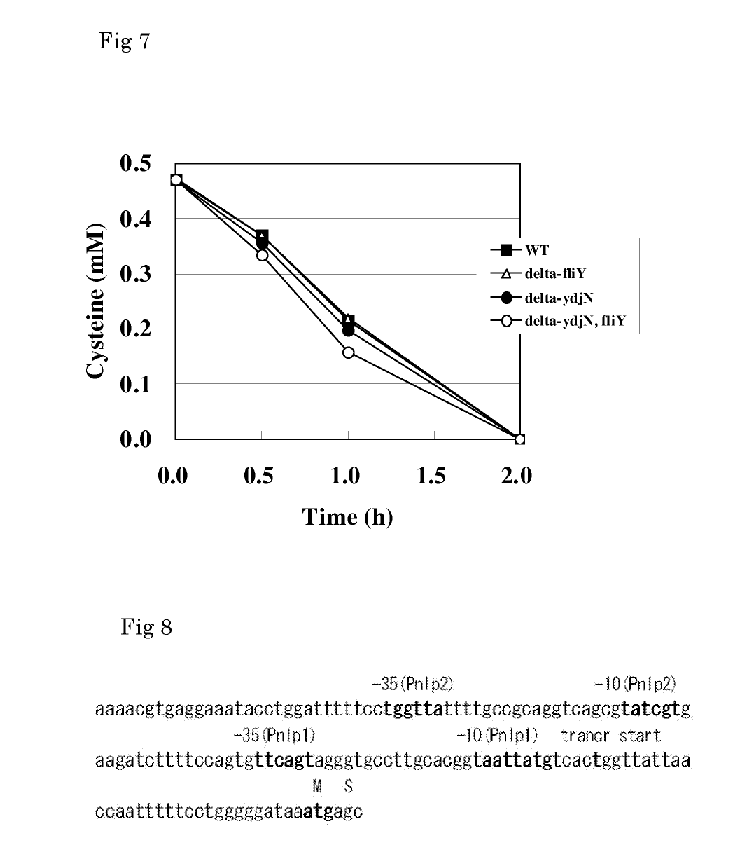

Although some references suggest the possibility that fliY may participate in the uptake of cysteine (Butler et al., Life Sci., 52, 1209-1215 (1993) etc.), this has not been directly or experimentally. In fact, the participation of FliY in the uptake of cysteine was not shown in this experiment. Furthermore, from the results of this experiment, it was predicted that even if FliY participates in uptake of cystine, it is not a highly active transporter, like YdjN. In addition, since the ydjN and fliY double deficient strain could grow with cystine as the sole cysteine source, it is considered that a transporter of cystine must exist besides these two transporters. Furthermore, although the cysteine uptake activity of the fliY-deficient strain was also examined, a significant difference, like that observed for ydjN, was not observed when compared with the non-deficient strain, and it was considered that FliY does not participate in the uptake of cysteine (FIG. 7). Alternatively, since cysteine gradually decreased in the medium (FIG. 7), uptake of cysteine into cells was expected, and it is supposed that a certain cysteine transporter (uptake system) exists in E. coli.

### Example 2

### Cysteine production by ydjN and/or fliY-Deficient E. coli

(1) Construction of Cysteine-Producing E. coli Strain

In order to impart the ability to produce cysteine to a ydjN- and/or fliY-deficient E. coli strain, a plasmid containing a mutant cysE coding for a mutant serine acetyltransferase with reduced feedback inhibition by L-cysteine (U.S. Patent Published Application No. 2005/0112731(A1)) was constructed. Specifically, a pACYC-DE1 plasmid was constructed according to the method for constructing pACYC-DES described in Japanese Patent Laid-open No. 2005-137369 (U.S. Patent Published Application No. 2005/0124049(A1), EP 1528108(A1)) except that the step of incorporating a mutant serA5 gene coding for a phosphoglycerate dehydrogenase desensitized to feedback inhibition by serine (described in U.S. Pat. No. 6,180,373) was omitted. While the plasmid pACYC-DES carried the aforementioned mutant serA5, the gene coding for the mutant SAT desensitized to feedback inhibition, the cysEX gene, and the ydeD gene coding for the L-cysteine and acetylserine secretion factor (U.S. Pat. No. 5,972,663), the plasmid pACYC-DE1 constructed above did not contain serA5, but contained cysEX and ydeD. To express all the genes, the ompA promoter was used.

Then, pACYC-DE1 was digested with Mnul and self-ligated to construct a plasmid in which about 330 by of the internal sequence of the ydeD gene ORF was deleted. This plasmid does not express YdeD (cysteine secretion factor), but carries only cysEX, and was designated pACYC-E1 and used for the following experiments. 5 strains, E. coli MG1655, MG1655ΔfliY, MG1655ΔfliY::Km, MG1655ΔydjN::Km, and MG1655ΔfliYΔydjN::Km, were transformed with pACYC-E1 to impart the ability to produce cysteineto each strain.

(2) Investigation of Effect of ydjN Deficiency and fliY Deficiency in E. coli on Cysteine Production

In order to investigate the effect of the ydjN and fliY deficiencies on the production of cysteine and cysteine-related compounds by fermentation, the fermentation culture was performed with cysteine-producing bacteria obtained by introducing pACYC-E1 into the MG1655 strain, ydjN or fliY-deficient strains, and ydjN and fliY-double deficient strain derived from the MG1655 strain, and amounts of cysteine and cysteine-related compounds that were produced were compared. For the culture, an E. coli cysteine production medium having the following composition was used.

E. coli Cysteine Production Medium (Concentrations of the Components are Final Concentrations):

Component 1:

| | | |
|---|---|---|
| (NH₄)₂SO₄ | 15 | g/L |
| KH₂PO₄ | 1.5 | g/L |
| MgSO₄•7H₂O | 1 | g/L |
| Tryptone | 10 | g/L |
| Yeast extract | 5 | g/L |
| NaCl | 10 | g/L |
| L-Histidine monohydrochloride | 135 | mg/L |
| monohydrate | | |
| L-Methionine | 300 | mg/L |

Component 2:

| | |
|---|---|
| Glucose | 40 g/L |

Component 3:

| | |
|---|---|
| Sodium thiosulfate | 7 g/L |

Component 4:

| | |
|---|---|
| Pyridoxine hydrochloride | 2 mg/L |

Component 5:

| | |
|---|---|
| Calcium carbonate | 20 g/L |

For these components, 100/47.5-fold (Component 1), 100/47.5-fold (Component 2), 50-fold (Component 3), and 1000-fold (Component 4) concentration stock solutions were prepared, and mixed upon use, and the volume of the mixture was adjusted to a predetermined volume with sterilized water to obtain the final concentrations. Sterilization was performed by autoclaving at 110° C. for 30 minutes (Components 1 and 2), hot air sterilization at 180° C. for 5 hours or longer (Component 5), and filter sterilization (Components 3 and 4).

The culture was performed according to the following procedures. The strains were each applied and spread on the LB agar medium, and precultured overnight at 37° C. Then, the cells corresponding to about 7 cm on the plate were scraped twice with an inoculating loop of 10 μl size (Blue Loop, NUNC), and inoculated into 2 ml of the aforementioned E. coli cysteine production medium contained in a large test tube (internal diameter: 23 mm, length: 20 cm). The amounts of the inoculated cells were adjusted so that the cell amounts at the time of the start of the culture are substantially the same. The culture was performed at 32° C. with shaking, and terminated after 40 hours. The cysteine that accumulated in the medium was quantified by the method described by Gaitonde, M. K. (Biochem. J., 1967 Aug., 104(2):627-33). Cysteine quantified above includes cystine, derivatives thereof such as S-sulfocysteine, thiazolidine derivatives and hemithioketals, or a mixture of them, in addition to cysteine, and the same shall apply to cysteine quantified below e, unless specified. Cysteine and other compounds quantified as described above may be described as L-cysteine related compounds. The experiment was performed six times for each strain, and averages and standard deviations for each are shown in Table 2.

TABLE 2

| | | Cysteine related |
|---|---|---|
| Strain | Genotype | compounds (g/L) |
| MG1655/pACYC-E1 | Wild-type | 0.056 ± 0.0055 |
| MG1655ΔfliY/pACYC-E1 | ΔfliY | 0.062 ± 0.0214 |
| MG1655ΔfliY::Km/pACYC-E1 | ΔfliY(::Km^{R}) | 0.082 ± 0.0172 |
| MG1655ΔydjN::Km/pACYC-E1 | ΔydjN(::Km^{R}) | 0.124 ± 0.0113 |
| MG1655ΔfliY | ΔfliY ΔydjN(::Km^{R}) | 0.273 ± 0.0381 |
| DydjN::Km/pACYC-E1 | | |

As shown in Table 2, strains lacking both fliY and ydjN were effective for increasing the production of the cysteine-related compounds. Moreover, the double deficiency of fliY and ydjN had a synergistic effect of markedly increasing the cysteine-related compounds as compared with a deficiency of each gene alone.

### Example 3

### Production of Cysteine by P. ananatis Deficient in ydjN and/or fliY

(1) Preparation of Cysteine-Producing P. ananatis EYPS1976(s) Strain

A cysteine-producing bacterium of P. ananatis was constructed by introducing cysE5 coding for a mutant serine acetyltransferase (U.S. Patent Published Application No. 2005/0112731), serA348 coding for a mutant 3-phosphoglycerate dehydrogenase (J. Biol. Chem., 1996, 271 (38):23235-8), and enhancing yeaS coding for a secretion factor for various amino acids (Japanese Patent Laid-open No. 2000-189180) and the cysPTWA cluster coding for a sulfur source uptake factor. The details of the construction method are described below.

(1-1) Introduction of CysE5 and YeaS into P. ananatis SC17 Strain

First, a plasmid for constructing the aforementioned strain was constructed. The method for it is described below.

By PCR using the chromosomal DNA of E. coli MG1655 (ATCC No. 47076) as the template as well as P1 (agctgagtcg acccccagga aaaattggtt aataac, SEQ ID NO: 30) and P2 (agctgagcat gcttccaact gcgctaatga cgc, SEQ ID NO: 31) as primers, a DNA fragment containing a promoter region of the nlpD gene (Pnlp0) of about 300 by was obtained. At the 5′ and 3′ ends of the aforementioned primers, sites for the restriction enzymes SalI and PaeI were designed, respectively. The PCR cycle was as follows: 95° C. for 3 minutes, then 2 cycles of 95° C. for 60 seconds, 50° C. for 30 seconds, and 72° C. for 40 seconds, 25 cycles of 94° C. for 20 seconds, 55° C. for 20 seconds, and 72° C. for 15 seconds, and 72° C. for 5 minutes as the final cycle. The obtained fragment was treated with SalI and PaeI, and inserted into pMIV-5JS (Japanese Patent Laid-open No. 2008-99668) at the SalI-PaeI site to obtain the plasmid pMIV-Pnlp0. The nucleotide sequence of the PaeI-SalI fragment of the Pnlp0 promoter inserted into this pMIV-Pnlp0 plasmid is as shown in SEQ ID NO: 27.

Then, by PCR using the chromosomal DNA of MG1655 as the template, as well as P3 (agctgatcta gaaaacagaa tttgcctggc ggc, SEQ ID NO: 32) and P4 (agctgaggat ccaggaagag tttgtagaaa cgc, SEQ ID NO: 33) as primers, a DNA fragment containing a terminator region of the rrnB gene of about 300 by was obtained. At the 5′ ends of the aforementioned primers, sites for the restriction enzymes XbaI and BamHI were designed, respectively. The PCR cycle was as follows: 95° C. for 3 minutes, then 2 cycles of 95° C. for 60 seconds, 50° C. for 30 seconds, and 72° C. for 40 seconds, 25 cycles of 94° C. for 20 seconds, 59° C. for 20 seconds, and 72° C. for 15 seconds, and 72° C. for 5 minutes as the final cycle. The obtained fragment was treated with XbaI and BamHI, and inserted into pMIV-Pnlp0 at the XbaI-BamHI site to obtain the plasmid pMIV-Pnlp0-ter.

Then, by PCR using the chromosomal DNA of the MG1655 strain as the template, as well as P5 (agctgagtcg acgtgttcgc tgaatacggg gt, SEQ ID NO: 34) and P6 (agctgatcta gagaaagcat caggattgca gc, SEQ ID NO: 35) as primers, a DNA fragment of about 700 by containing the yeaS gene was obtained. At the 5′ ends of the aforementioned primers, sites for the restriction enzymes SalI and XbaI were designed, respectively. The PCR cycle was as follows: 95° C. for 3 minutes, then 2 cycles of 95° C. for 60 seconds, 50° C. for 30 seconds, and 72° C. for 40 seconds, 25 cycles of 94° C. for 20 seconds, 55° C. for 20 seconds, and 72° C. for 15 seconds, and 72° C. for 5 minutes as the final cycle. The obtained fragment was treated with SalI and XbaI, and inserted into pMIV-Pnlp0-ter at the SalI-XbaI site to obtain the plasmid pMIV-Pnlp0-YeaS3. As described above, a yeaS expression unit including the pMIV-5JS vector on which, in order, the nlpD promoter, the yeaS gene, and the rrnB terminator were ligated was constructed.

In order to modify the −10 region of the nlpD promoter to make it a stronger promoter, the −10 region was randomized by the following method. The nlpD promoter region contains two regions presumed to function as promoters (FIG. 8), and they are indicated as pnlp1 and pnlp2, respectively, in the drawing. By PCR using the plasmid pMIV-Pnlp0 as the template as well as P1 and P7 (atcgtgaaga tcttttccag tgttnannag ggtgccttgc acggtnatna ngtcactgg (“n” means that the corresponding residue can be any of a, t, g and c), SEQ ID NO: 36) as primers, a DNA fragment in which the −10 region at the 3′ end sequence of the nlpD promoter (referred to as −10(Pnlp1)) was randomized was obtained. The PCR cycle was as follows: 95° C. for 3 minutes, then 2 cycles of 95° C. for 60 seconds, 50° C. for 30 seconds, and 72° C. for 40 seconds, 25 cycles of 94° C. for 20 seconds, 60° C. for 20 seconds, and 72° C. for 15 seconds, and 72° C. for 5 minutes as the final cycle.

Furthermore, by PCR using the plasmid pMIV-Pnlp0 as the template as well as P2 and P8 (tggaaaagat cttcannnnn cgctgacctg cg (“n” means that the corresponding residue can be any of a, t, g and c), SEQ ID NO: 37) as primers, a DNA fragment in which the −10 region at the 5′ end sequence of the nlpD promoter (referred to as −10(Pnlp2)) was randomized was similarly obtained (FIG. 1). The PCR cycle was as follows: 95° C. for 3 minutes, then 2 cycles of 95° C. for 60 seconds, 50° C. for 30 seconds, and 72° C. for 40 seconds, 25 cycles of 94° C. for 20 seconds, 60° C. for 20 seconds, and 72° C. for 15 seconds, and 72° C. for 5 minutes as the final cycle.

The obtained 3′ and 5′ end fragments could be ligated using the BglII sites designed in the primers P7 and P8, and the full length of the nlpD promoter in which two −10 regions were randomized could be constructed by such ligation. By PCR using this fragment as the template as well as P1 and P2 as primers, a DNA fragment corresponding to a modified type nlpD promoter of the full length was obtained. The PCR cycle was as follows: 95° C. for 3 minutes, then 2 cycles of 95° C. for 60 seconds, 50° C. for 30 seconds, and 72° C. for 40 seconds, 12 cycles of 94° C. for 20 seconds, 60° C. for 20 seconds, and 72° C. for 15 seconds, and 72° C. for 5 minutes as the final cycle.

The amplified fragment was treated with the restriction enzymes SalI and Pad, for which sites were designed in the 5′ ends of the primers, and inserted into the plasmid pMIV-Pnlp0-YeaS3 which had been similarly treated with SalI and Pad to substitute the mutant Pnlp for the wild-type nlpD promoter region (Pnlp0) on the plasmid. From such plasmids, one having the promoter sequence (Pnlp8) shown in SEQ ID NO: 28 was selected, and designated pMIV-Pnlp8-YeaS7 (the nucleotide sequence of the Pad-SalI fragment of the Pnlp8 promoter inserted into this plasmid was as shown in SEQ ID NO: 28). In the same manner, a DNA fragment of the nlpD promoter region containing a mutation was inserted into the plasmid pMIV-Pnlp0-ter treated with SalI and Pad to substitute the mutant Pnlp for the nlpD promoter region (region of Pnlp0) on the plasmid. One of them was designated pMIV-Pnlp23-ter. The nucleotide sequence of the Pad-SalI fragment of the Pnlp23 promoter inserted into this plasmid was as shown in SEQ ID NO: 29.

Then, from pMW-Pomp-cysE5 (WO2005/007841), the Pomp-cysE5 cassette portion was excised with Pad and Sad, and inserted into the same site of pMIV-5JS to construct pMIV-Pomp-CysE5. pMW-Pomp-cysE5 was obtained by inserting the cysE5 gene coding for the mutant SAT ligated with the ompC gene promoter into pMW118. From pACYC184 (GenBank/EMBL accession number X06403, available from NIPPON GENE), the tetracycline resistance gene was excised with XbaI and Eco88I, and this gene fragment was treated with the Klenow fragment, and then inserted into pMIV-Pomp-CysE5 at the PvuI site to construct pMT-Pomp-CysE5. Then, pMIV-Pnlp8-YeaS7 was digested with HindIII, blunt-ended with the Klenow fragment, and then digested with NcoI to excise a fragment containing the cassette of the Pnlp8-YeaS-rrnB terminator and the chloramphenicol resistance marker. This fragment was ligated with a SmaI and NcoI digestion fragment of pMT-Pomp-CysE5 similarly having pMIV-5JS as the backbone to construct pMT-EY2. pMT-EY2 is a plasmid having the Pnlp8-YeaS-rmB terminator cassette and the Pomp-CysE5 cassette on one plasmid.

pMT-EY2 described above has the attachment sites of Mu phage originated from pMIV-5JS (Japanese Patent Laid-open No. 2008-99668). By allowing this plasmid to coexist with the helper plasmid pMH10 having Mu transposase (Zimenkov D. et al., Biotechnologiya and (in Russian), 6, 1-22 (2004)) in the same cell, the cassette of PompC-cysE5-Pnlp8-YeaS-rrnB terminator including the chloramphenicol resistance marker located between the attachment sites of Mu phage on this pMT-EY2 plasmid can be inserted into the chromosome of the P. ananatis SC17 strain (U.S. Pat. No. 6,596,517). Furthermore, since the chloramphenicol resistance marker located on the pMT-EY2 plasmid exists between two attachment sites of λ phage (λattR and λattL), the chloramphenicol resistance marker can be excised and removed by the method described later.

First, an SC17 strain introduced with pMH10 by electroporation was selected by overnight culture at 30° C. on the LB agar medium containing 20 mg/L of kanamycin. The obtained transformant was cultured at 30° C., and pMT-EY2 was further introduced into this strain by electroporation. This strain transformed with both pMH10 and pMT-EY2 was given a heat shock at 42° C. for 20 minutes, and colonies of chloramphenicol-resistant strains were selected on the LB agar medium containing 20 mg/L of chloramphenicol. The culture temperature for this selection was 39° C. As described above, about 50 clones were obtained, and the curing of pMH10 and pMT-EY2 was performed by culturing each clone at 39° C. for 48 hours on the LB agar medium. A strain showing chloramphenicol resistance due to the insertion of the cassette on the chromosome and showing kanamycin and ampicillin sensitivities due to the curing of both plasmids was obtained. Furthermore, it was confirmed that the objective cassette was inserted into the chromosome of the obtained strain by PCR using the chromosomal DNA of this strain as the template as well as P1 and P6 as primers. All the obtained clones were designated EY01 to EY50, respectively, and L-cysteine production culture was performed by using the EY01 to EY50 strains as described below. The EY19 strain was selected, which produced L-cysteine in the largest amount as a result of the culture.

An L-cysteine production medium (composition: 15 g/L of ammonium sulfate, 1.5 g/L of potassium dihydrogenphosphate, 1 g/L of magnesium sulfate heptahydrate, 0.1 g/L of tryptone, 0.05 g/L of yeast extract, 0.1 g/L sodium chloride, 20 g/L of calcium carbonate, 40 g/L of glucose, and 20 mg/L of tetracycline) was used for the culture.

The L-cysteine production culture was performed by the following procedure. The SC17/pMT-PompCysE5 strain and SC17/pMT-EY2 strain were each applied on LB agar medium and precultured overnight at 34° C., then cells corresponding to ⅛ of the plate were scraped with an inoculation loop, inoculated into 2 ml of the L-cysteine production medium contained in a large test tube (internal diameter: 23 mm, length: 20 cm), and cultured at 32° C. with shaking at 220 to 230 rpm, and the culture was terminated after two days.

The chloramphenicol resistance marker introduced into the EY19 strain was removed with an excision system derived from λ phage. Specifically, the EY19 strain was transformed with pMT-Int-Xis2 (WO2005/010175) carrying the Int-Xis gene of λ phage, and an EY19(s) strain showing chloramphenicol sensitivity was obtained from the obtained transformants. Examples of removal of a marker using the excision system derived from phage are described in detail in Japanese Patent Laid-open No. 2005-058227, WO2007/119880, and so forth.

(1-2) Preparation of cysPTWA Gene Expression-Enhanced Strain from EY19(s) Strain

Then, in order to enhance expression of the cysPTWA gene, the promoter located upstream of the cysPTWA gene cluster on the chromosome was replaced with the aforementioned potent promoter Pnlp8. A DNA fragment containing the nlp8 promoter of about 300 by was obtained by PCR using pMIV-Pnlp8-YeaS7 as the template as well as P1 and P2 as primers. The PCR cycle was as follows: 95° C. for 3 minutes, then 2 cycles of 95° C. for 60 seconds, 50° C. for 30 seconds, and 72° C. for 40 seconds, 20 cycles of 94° C. for 20 seconds, 59° C. for 20 seconds, and 72° C. for 15 seconds, and 72° C. for 5 minutes as the final cycle.

The amplified DNA fragment containing the nlp8 promoter was treated with the Klenow fragment, inserted into the plasmid pMW118-(λattL-KmR-λattR) (WO2006/093322A2), digested with XbaI, and then treated with the Klenow fragment to obtain the plasmid pMW-Km-Pnlp8. By PCR using pMW-Km-Pnlp8 as a template as well as P9 (tccgctcacg atttttttca tcgctggtaa ggtcatttat cccccaggaa aaattggtta, SEQ ID NO: 38) and P10 (tttcacaccg ctcaaccgca gggcataacc ggcccttgaa gcctgctttt ttatactaag ttg, SEQ ID NO: 39) as primers, a DNA fragment of about 1.6 kb containing the Km-Pnlp8 cassette was amplified. The PCR cycle for this amplification was as follows: 95° C. for 3 minutes, then 2 cycles of 95° C. for 60 seconds, 50° C. for 30 seconds, and 72° C. for 40 seconds, 30 cycles of 94° C. for 20 seconds, 54° C. for 20 seconds, and 72° C. for 90 seconds, and 72° C. for 5 minutes as the final cycle. For both of the primers, a sequence that acts as a target on the chromosome for inserting an objective fragment by λ-dependent integration (“Red-driven integration” (Proc. Natl. Acad. Sci. USA, 2000, vol. 97, No. 12, pp. 6640-6645)) (in this case, a sequence near the promoter of cysPTWA) was designed. Therefore, if the obtained DNA fragment is inserted into the objective strain by this λ-dependent integration, Km-Pnlp8 is inserted immediately before the cysPTWA gene on the chromosome, and the cysPTWA gene is ligated with the nlp8 promoter. The nucleotide sequence of the cysPTWA gene cluster is shown in SEQ ID NO: 19, and the amino acid sequences encoded by the cysP, cysT and cysW genes are shown in SEQ ID NOS: 20 to 22, respectively. The nucleotide sequence of the cysA gene and the amino acid sequence encoded by this gene are shown in SEQ ID NOS: 23 and 24, respectively.

The P. ananatis SC17(0)/RSF-Red-TER strain is a host strain for efficiently performing the λ-dependent integration, and was obtained by introducing the helper plasmid RSF-Red-TER which expresses the gam, bet and exo genes of λ (henceforth referred to as “λ, Red genes”) into the SC17(0) strain, which is a λ Red gene product-resistant P. ananatis strain (WO2008/075483). A method for constructing the RSF-Red-TER plasmid is disclosed in detail in WO2008/075483.

The aforementioned SC17(0)/RSF-Red-TER strain was cultured with IPTG to induce expression of λ Red genes and prepare cells for electroporation. The aforementioned objective DNA fragment was introduced into these cells by electroporation, and a recombinant strain into which the nlp8 promoter was inserted upstream of the cysPTWA gene by λ-dependent integration was obtained by using kanamycin resistance as a marker. By PCR using the chromosomal DNA of the obtained strain as the template, as well as P11 (ctttgtccct ttagtgaagg, SEQ ID NO: 40) and P12 (agctgatcta gaagctgact cgagttaatg gcctcccaga cgac, SEQ ID NO: 41) as primers, it was confirmed that the objective structure, Km-Pnlp8-cysPTWA, was formed, and this strain was designated SC17(0)-Pnlp8-PTWA.

Then, the chromosomal DNA of the SC17(0)-Pnlp8-PTWA strain was purified, and 10 μg of this chromosomal DNA was introduced into the EY19(s) strain by electroporation to obtain a kanamycin-resistant strain. Amplification was performed by PCR using the chromosomal DNA of the obtained strain as the template as well as P11 and P12 as primers to confirm that the structure of Km-Pnlp8-cysPTWA had been introduced into the chromosome of the EY19(s) strain. The strain obtained as described above was designated EYP197. Furthermore, the kanamycin resistance marker was removed from the chromosome by using pMT-Int-Xis2 as described above, and the strain that became kanamycin sensitive was designated EYP197(s).

(1-3) Preparation of an EYP197(s) Strain Having a Mutant 3-Phosphoglycerate Dehydrogenase (serA348) Gene

The serA348 gene encodes 3-phosphoglycerate dehydrogenase of Pantoea ananatis, but includes a mutation resulting in substitution of an alanine residue for the asparagine residue at the 348th position (N348A) (J. Biol. Chem., 1996, 271 (38):23235-8), and was constructed by the following method.

The sequence of the wild-type serA gene derived from Pantoea ananatis and the amino acid sequence are shown in SEQ ID NOS: 17 and 18, respectively. In order to obtain a 3′-end side DNA fragment of the serA gene into which the aforementioned mutation was introduced, PCR was performed by using the chromosomal DNA of the SC17 strain as the template as well as P13 (agctgagtcg acatggcaaa ggtatcactg gaa, SEQ ID NO: 42) and P14 (gagaacgccc gggcgggctt cgtgaatatg cagc, SEQ ID NO: 43) as primers (95° C. for 3 minutes, then 2 cycles of 95° C. for 60 seconds, 50° C. for 30 seconds, and 72° C. for 40 seconds, 25 cycles of 94° C. for 20 seconds, 60° C. for 20 seconds, and 72° C. for 60 seconds, and 72° C. for 5 minutes as the final cycle). Then, in order to obtain a 5′-end side DNA fragment into which the mutation was introduced, PCR was performed in the same manner by using the chromosomal DNA of the SC17 strain as the template as well as P15 (agctgatcta gacgtgggat cagtaaagca gg, SEQ ID NO: 44) and P16 (aaaaccgccc gggcgttctc ac, SEQ ID NO: 45) as primers (95° C. for 3 minutes, then 2 cycles of 95° C. for 60 seconds, 50° C. for 30 seconds, and 72° C. for 40 seconds, 20 cycles of 94° C. for 20 seconds, 60° C. for 20 seconds, and 72° C. for 20 seconds, and 72° C. for 5 minutes as the final cycle). Both of the obtained PCR fragments were treated with the restriction enzyme SmaI, and ligated by using a DNA ligase to obtain a DNA fragment corresponding to a full-length mutant serA gene including the desired mutation (N348A). This DNA fragment was amplified by PCR using it as the template as well as P13 and P15 as primers (95° C. for 3 minutes, then 2 cycles of 95° C. for 60 seconds, 50° C. for 30 seconds, and 72° C. for 40 seconds, 15 cycles of 94° C. for 20 seconds, 60° C. for 20 seconds, and 72° C. for 75 seconds, and 72° C. for 5 minutes as the final cycle). The SalI and the XbaI restriction enzyme sites designed in the P13 and P15 primers were treated with SalI and XbaI, and the fragment was inserted into pMIV-Pnlp8-ter similarly treated with SalI and XbaI to prepare pMIV-Pnlp8-serA348.

The pMIV-Pnlp8-serA348 included the attachment site of Mu originating in pMIV-5JS (Japanese Patent Laid-open No. 2008-99668). By using this plasmid together with the helper plasmid pMH10 having Mu transposase, the cassette of Pnlp8-serA348-rrnB terminator including the chloramphenicol resistance marker can be inserted into the chromosome of the P. ananatis SC17 strain, as described above. The pMIV-Pnlp8-serA348 plasmid and pMH10 were introduced into the SC17(0) strain to obtain a strain in which the cassette of Pnlp8-serA348-rrnB terminator was inserted into the chromosome. By PCR using the primers P1 and P15, it was confirmed that the objective cassette was present in the cells. The 3-phosphoglycerate dehydrogenase activity in about 50 cell extracts of the obtained clones was measured, and the strain which showed the highest activity was selected, and designated SC17int-serA348. Then, 10 μg of the chromosomal DNA of the SC17int-serA348 strain was introduced into the EYP197(s) strain by electroporation to obtain a chloramphenicol-resistant strain, and by PCR using the primers P1 and P15, it was confirmed that the structure of Pnlp8-serA348 had been introduced together with the chloramphenicol resistance marker into the chromosome of the EYP197(s) strain. The strain obtained as described above was designated EYPS1976. By the aforementioned method for removing a marker using pMT-Int-Xis2, the chloramphenicol resistance marker was removed, and the strain that became chloramphenicol-sensitive was designated EYPS1976(s).

(2) Construction of ydjN- and/or fliY-Deficient Cysteine-Producing Bacteria

Strains deficient in ydjN and/or fliY were constructed from the EYPS1976(s) strain. A ydjN gene-deficient strain and a fliY region-deficient strain were prepared by λ-dependent integration using the aforementioned P. ananatis SC17(0)/RSF-Red-TER strain as a host bacterium.

The yecS gene (nucleotide sequence: SEQ ID NO: 11, amino acid sequence: SEQ ID NO: 12) and the yecC gene (nucleotide sequence: SEQ ID NO: 13, amino acid sequence: SEQ ID NO: 14) are present downstream from the fliY gene, and may possibly form an operon and function as an ABC transporter. yecS and yecC may form one transcription unit (http://ecocyc.org/). Therefore, for the fliY deficiency, all three of these genes (fliY flanking regions) were deleted.

To obtain a DNA fragment for the ydjN gene-deficient strain, primers DydjN(Pa)-F (acctctgctg ctctcctgac cagggaatgc tgcattacat cggagttgct tgaagcctgc ttttttatac taagttggca, SEQ ID NO: 46) DydjN(Pa)-R (agacaaaaac agagagaaag acctggcggt gtacgccagg tctggcgtga cgctcaagtt agtataaaaa agctgaacga, SEQ ID NO: 47) were used, and to obtain a DNA fragment for the fliY region-deficient strain, primers DfliY-FW (atggctttct cacagattcg tcgccaggtg gtgacgggaa tgatggcggt tgaagcctgc ttttttatac taagttggca, SEQ ID NO: 48) and DyecC-RV (ttacgccgcc aacttctggc ggcaccgggt ttattgatta agaaatttat cgctcaagtt agtataaaaa agctgaacga, SEQ ID NO: 49) were used. As the template, pMW118-(λattL-Km^{r}-λattR) (WO2006/093322A2) (see above) was used, and PCR was performed at 94° C. for 5 minutes, followed by 30 cycles of 94° C. for 30 seconds, 55° C. for 30 seconds and 72° C. for 2 minutes and 30 seconds to obtain a DNA fragment containing Km^{r} between the homologous sequences used for the recombination.

Each of the DNA Fragments was Introduced into the P. ananatis SC17(0)/RSF-Red-TER strain by electroporation to construct SC17(0)ΔydjN::Km strain (the λattL-Kmr-λattR fragment was inserted into the ydjN gene region) and the SC17(0)ΔfliY::Km strain (the λattL-Kmr-λattR fragment was inserted into the fliY gene region). Then, the EYPS1976(s) strain was transformed with the chromosomal DNAs prepared from the SC17(0)ΔydjN::Km strain and the SC17(0)ΔfliY::Km strain to obtain strains deficient in each gene, the EYPSΔydjN::Km and EYPSΔfliY::Km strains, from the EYPS1976(s) strain. Furthermore, pMT-Int-Xis2 (WO2005/010175) mentioned above was introduced into the EYPSΔfliY::Km strain, and the kanamycin resistance gene was excised by using the excisive system derived from phage to obtain kanamycin-sensitive EYPSΔfliY strain. Then, the EYPSΔfliY strain was transformed with chromosomal DNA prepared the from the SC17(0)ΔydjN::Km strain to obtain a double deficient strain, EYPSΔfliYΔydjN::Km strain. In this manner, the ydjN gene-deficient strain, the fliY region-deficient strains, and the double deficient strain for these genes based on the cysteine-producing bacterium, EYPS1976(s) strain, were constructed.

(3) Investigation of the Effect of the ydjN and fliY Deficiencies in P. ananatis on Cysteine Production

Culture for fermentative production was performed with the ydjN gene-deficient strain, the fliY region-deficient strains, and the double deficient strain for these genes obtained above, and production amounts of the cysteine-related compounds were compared. For the culture, a P. ananatis cysteine production medium having the following composition was used.

P. ananatis Cysteine Production Medium (Concentration of the Components are Final Concentrations):

Component 1:

| | | |
|---|---|---|
| (NH₄)₂SO₄ | 15 | g/L |
| KH₂PO₄ | 1.5 | g/L |
| MgSO₄•7H₂O | 1 | g/L |
| Thiamine hydrochloride | 0.1 | mg/L |

Component 2:

| | | |
|---|---|---|
| FeSO₄•7H₂O | 1.7 | mg/L |
| Na₂MoO₄•2H₂O | 0.15 | mg/L |
| CoCl₂•6H₂O | 0.7 | mg/L |
| MnCl•4H₂O | 1.6 | mg/L |
| ZnSO₄•7H₂O | 0.3 | mg/L |
| CuSO₄•5H₂O | 0.25 | mg/L |

Component 3:

| | |
|---|---|
| Tryptone | 0.6 g/L |
| Yeast extract | 0.3 g/L |
| NaCl | 0.6 g/L |

Component 4:

| | |
|---|---|
| Calcium carbonate | 20 g/L |

Component 5:

| | |
|---|---|
| L-Histidine monohydrochloride | 135 mg/L |
| monohydrate | |

Component 6:

| | |
|---|---|
| Sodium thiosulfate | 6 g/L |

Component 7:

| | |
|---|---|
| Pyridoxine hydrochloride | 2 mg/L |

Component 8:

| | |
|---|---|
| Glucose | 40 g/L |

For these components, 10-fold (Component 1), 1000-fold (Component 2), 100/6-fold (Component 3), 100-fold (Component 5), 350/6-fold (Component 6), 1000-fold (Component 7) and 10-fold (Component 8) concentration stock solutions were prepared, and mixed upon use, and the volume of the mixture was adjusted to a predetermined volume with sterilized water to obtain the final concentrations. Sterilization was performed by autoclaving at 110° C. for 30 minutes (Components 1, 2, 3, 5 and 8), hot air sterilization at 180° C. for 5 hours or longer (Component 4), and filter sterilization (Components 6 and 7).

The culture was performed according to the following procedures. The strains were each applied and spread on the LB agar medium, and precultured overnight at 34° C. Then, the cells corresponding to about 7 cm on the plate were scraped twice with an inoculating loop of 10 μl size (Blue Loop, NUNC), and inoculated into 2 ml of the aforementioned P. ananatis cysteine production medium contained in a large test tube (internal diameter: 23 mm, length: 20 cm). The amounts of inoculated cells were adjusted so that the cell amounts at the time of the start of the culture were substantially the same.

The culture was performed at 32° C. with shaking, and terminated after 43 hours. At this time, complete consumption of glucose in the medium was confirmed. The quantification of cysteine which had accumulated in the medium was performed by the method described by Gaitonde, M. K. (Biochem. J., 1967 Aug., 104(2):627-33). The experiment was performed six times for each of the strains, and averages and standard deviations of the results are shown in Table 3. For the strain deficient in only fliY, the amount of cysteine-related compounds did not increase, and for the strain deficient in only ydjN, the amount of cysteine-related compounds slightly increased. Furthermore, for the double deficient strain for fliY and ydjN, the amount of cysteine-related compounds increased to a much higher degree as compared with that when only one gene was deficient. It was found that ydjN deficiency in P. ananatis was effective for the production of the cysteine-related compounds, and when further combined with the fliY deficiency, a synergistic effect was observed.

TABLE 3

| | | Cysteine related compounds |
|---|---|---|
| Strain | Genotype | (g/L) |
| EYPS1976(s) | Wild-type | 0.80 ± 0.031 |
| EYPSΔfliY | ΔfliY | 0.71 ± 0.046 |
| EYPSΔydjN::Km | ΔydjN(::Km^{R}) | 0.92 ± 0.069 |
| EYPSΔfliYΔydjN::Km | ΔfliY ΔydjN(::Km^{R}) | 1.78 ± 0.081 |

Explanation of Sequence Listing

SEQ ID NO: 1: Nucleotide sequence of ydjN gene of Escherichia coli

SEQ ID NO: 2: Amino acid sequence of YdjN of Escherichia coli

SEQ ID NO: 3: Nucleotide sequence of ydjN gene of Pantoea ananatis

SEQ ID NO: 4: Amino acid sequence of YdjN of Pantoea ananatis

SEQ ID NO: 5: Nucleotide sequence of fliY gene of Escherichia coli

SEQ ID NO: 6: Amino acid sequence of FliY of Escherichia coli

SEQ ID NO: 7: Nucleotide sequence of fliY gene of Pantoea ananatis

SEQ ID NO: 8: Amino acid sequence of FliY of Pantoea ananatis

SEQ ID NO: 9: Nucleotide sequence of cysE gene of Escherichia coli

SEQ ID NO: 10: Amino acid sequence of SAT encoded by cysE gene of Escherichia coli

SEQ ID NO: 11: Nucleotide sequence of yecS gene of Pantoea ananatis SEQ ID NO: 12: Amino acid sequence encoded by yecS gene of Pantoea ananatis

SEQ ID NO: 13: Nucleotide sequence of yecC gene of Pantoea ananatis

SEQ ID NO: 14: Amino acid sequence encoded by yecC gene of Pantoea ananatis

SEQ ID NO: 15: Nucleotide sequence of yeaS gene of Escherichia coli

SEQ ID NO: 16: Amino acid sequence encoded by yeaS gene of Escherichia coli SEQ ID NO: 17: Nucleotide sequence of serA gene of Pantoea ananatis

SEQ ID NO: 18: Amino acid sequence encoded by serA gene of Pantoea ananatis

SEQ ID NO: 19: Nucleotide sequence of cysPTWA gene cluster

SEQ ID NO: 20: Amino acid sequence encoded by cysP gene

SEQ ID NO: 21: Amino acid sequence encoded by cysT gene

SEQ ID NO: 22: Amino acid sequence encoded by cysW gene

SEQ ID NO: 23: Nucleotide sequence of cysA gene

SEQ ID NO: 24: Amino acid sequence encoded by cysA gene

SEQ ID NO: 25: Nucleotide sequence of cysM gene

SEQ ID NO: 26: Amino acid sequence encoded by cysM gene

SEQ ID NO: 27: Nucleotide sequence of Pnlp0

SEQ ID NO: 28: Nucleotide sequence of Pnlp8

SEQ ID NO: 29: Nucleotide sequence of Pnlp23

SEQ ID NOS: 30 to 45: Primers P1 to P16

SEQ ID NO: 46: Nucleotide sequence of primer DydjN(Pa)-F

SEQ ID NO: 47: Nucleotide sequence of primer DydjN(Pa)-R

SEQ ID NO: 48: Nucleotide sequence of primer DfliY-FW

SEQ ID NO: 49: Nucleotide sequence of primer DyecC-RV

SEQ ID NO: 50: Primer DcysE(Ec)-F

SEQ ID NO: 51: Primer DcysE(Ec)-R

SEQ ID NO: 52: Primer DydjN(Ec)-F

SEQ ID NO: 53: Primer DydjN(Ec)-R

SEQ ID NO: 54: Primer ydjN(Ec)-SalIFW2

SEQ ID NO: 55: Primer ydjN(Ec)-xbaIRV2

SEQ ID NO: 56: Primer ydjN2(Pa)-SalIFW

SEQ ID NO: 57: Primer ydjN2(Pa)-xbaIRV

SEQ ID NO: 58: Primer DfliY(Ec)-FW

SEQ ID NO: 59: Primer DfliY(Ec)-RV

SEQ ID NO: 60: Primer fliY(Ec)SalI-F

SEQ ID NO: 61: Primer fliY(Ec)XbaI-R

SEQ ID NO: 62: Nucleotide sequence of the promoter Pnlp.

While the invention has been described in detail with reference to exemplary embodiments thereof, it will be apparent to one skilled in the art that various changes can be made, and equivalents employed, without departing from the scope of the invention. Each of the aforementioned documents is incorporated by reference herein in its entirety.

What is claimed is:

1. A bacterium belonging to the family Enterobacteriaceae, which is able to produce L-cysteine, and has been modified to decrease the activity of the YdjN protein.

2. The bacterium according to claim 1, wherein the YdjN protein has the amino acid sequence of SEQ ID NO: 2 or 4, or a variant thereof.

3. The bacterium according to claim 1, which has been further modified to decrease the activity of the FliY protein.

4. The bacterium according to claim 3, wherein the FliY protein has the amino acid sequence of SEQ ID NO: 6 or 8, or a variant thereof.

5. The bacterium according to claims 1, wherein the activity of the YdjN or FliY protein has been decreased by a method selected from the group consisting of
a) reducing expression of the ydjN or fliY gene,
b) disrupting the ydjN or fliY gene, and
c) combinations thereof.

6. The bacterium according to claim 5, wherein the ydjN gene is selected from the group consisting of:
(a) a DNA comprising the nucleotide sequence of SEQ ID NO: 1 or 3,
(b) a DNA which is able to hybridize with a sequence complementary to the nucleotide sequence of SEQ ID NO: 1 or 3, or a probe which is prepared from the nucleotide sequence, under stringent conditions, and
(c) a DNA which has a homology of 95% or more to the nucleotide sequence of SEQ ID NO: 1 or 3.

7. The bacterium according to claim 5, wherein the fliY gene is selected from the group consisting of:
(d) a DNA comprising the nucleotide sequence of SEQ ID NO: 5 or 7,
(e) a DNA which is able to hybridize with a sequence complementary to the nucleotide sequence of SEQ ID NO: 5 or 7, or a probe which is prepared from the nucleotide sequence, under stringent conditions, and
(f) a DNA which has a homology of 95% or more to the nucleotide sequence of SEQ ID NO: 5 or 7.

8. The bacterium according to claim 1, which further has at least one of the following characteristics:
i) it has been modified to increase serine acetyltransferase activity,
ii) it has been modified to increase expression of the yeaS gene,
iii) it has been modified to increase 3-phosphoglycerate dehydrogenase activity,
iv) it has been modified to enhance activity of the sulfate/thio sulfate transport system.

9. The bacterium according to claim 1, which belongs to the genus Pantoea.

10. The bacterium according to claim 9, which is Pantoea ananatis.

11. The bacterium according to claim 1, which is Escherichia coli.

12. A method for producing a product selected from the group consisting of L-cysteine, L-cystine, a derivative or precursor of L-cysteine or L-cystine, and combinations thereof, which comprises culturing the bacterium according to claim 1 in a medium and collecting the product from the medium.


SEQUENCE LISTING (62 sequences)

SEQ ID NO: 1 - DNA, 1392 bp, Escherichia coli
  CDS (1)..(1392)
atgaactttccattaattgcgaacatcgtggtgttcgttgtactgctgtttgcgctggctcagacccgccataaacagtggagtctggcgaaaaaagtgctggtgggtctggtgatgggtgtggtttttggccttgccctgcataccatttatggttctgacagccaggtacttaaagattctgtacagtggtttaacatcgttggtaacggctatgttcaactgctgcaaatgatcgttatgccgttagtcttcgcctctattctgagcgcggttgcccgtctgcataacgcatctcagttaggcaaaatcagttttctgaccatcggtacgcttttgtttaccacgctgattgcggcgctggtcggtgtgctggtcaccaacctgtttggtttgacggctgaaggtctggttcagggtggtgcagaaactgcacgtctgaacgccattgaaagtaactatgttggtaaagtctctgatctgagcgttccgcagctggtcttgtcctttatcccgaaaaacccgtttgccgatcttaccggagccaatccgacgtcaattatcagcgtggtaatttttgccgcattcctcggcgtagctgcattgaaactgctgaaggatgatgcgccgaaaggtgaacgcgtcttagccgctatcgataccctacaaagctgggtgatgaaactggttcgcctggtcatgcagttgaccccttacggcgttctggctctaatgaccaaagtggttgcaggttctaacttgcaagacatcatcaaactgggaagtttcgttgtcgcgtcctacctcggtctgctgattatgtttgcagtgcatggcattctgctgggcattaatggcgtgagtccgctgaagtacttccgtaaggtatggcctgtgctgacgtttgcctttaccagccgttccagtgctgcgtctatcccactgaatgtggaagcacaaacgcgtcgtctgggcgttcctgaatccatcgccagtttcgccgcctctttcggtgcaaccattggtcagaacggctgcgccggtttgtatccggcaatgctggcggtgatggttgcgcctacggttggcattaacccgctggacccgatgtggattgcgacgctggtcggtattgttaccgttagttccgcaggcgttgccggtgtcggtggtggtgcaactttcgccgcactgattgtactgcctgcgatgggcctgccagtaaccctggtggcgctgttaatctccgttgaaccgcttatcgacatgggccgtacggcgttaaacgttagtggctcgatgacagctggcacgctgaccagccagtggctgaagcaaaccgataaagccattctggatagcgaagacgacgccgaactggcacaccattaa

SEQ ID NO: 2 - protein, 463 aa, Escherichia coli
MNFPLIANIVVFVVLLFALAQTRHKQWSLAKKVLVGLVMGVVFGLALHTIYGSDSQVLKDSVQWFNIVGNGYVQLLQMIVMPLVFASILSAVARLHNASQLGKISFLTIGTLLFTTLIAALVGVLVTNLFGLTAEGLVQGGAETARLNAIESNYVGKVSDLSVPQLVLSFIPKNPFADLTGANPTSIISVVIFAAFLGVAALKLLKDDAPKGERVLAAIDTLQSWVMKLVRLVMQLTPYGVLALMTKVVAGSNLQDIIKLGSFVVASYLGLLIMFAVHGILLGINGVSPLKYFRKVWPVLTFAFTSRSSAASIPLNVEAQTRRLGVPESIASFAASFGATIGQNGCAGLYPAMLAVMVAPTVGINPLDPMWIATLVGIVTVSSAGVAGVGGGATFAALIVLPAMGLPVTLVALLISVEPLIDMGRTALNVSGSMTAGTLTSQWLKQTDKAILDSEDDAELAHH

SEQ ID NO: 3 - DNA, 1392 bp, Pantoea ananatis
  CDS (1)..(1392)
atggatattcctcttacgcttaacgtcgtggcatttgtgctgctactgcttttgctgtcacgccttggccgggcaaactggagcctgtcgaaaaaggttctgactggcctggtgctgggtgtggtgtttggcctcgcgctccagctgatttatggcggcaacagcgatatcgttaaagcctctatcggctggtttaacatcgtgggtaacggttatgttcagctgttacagatgattgtgatgccgctggtgttcgtgtcgattctcagcgccgtggcccgcctgcacaatgcctcctcactggggaaaatcagttttctgaccatcggcgtactgctgtttacaacggcaatttctgccctgatcggcgtttttgtcaccggcctgtttcaccttaacgcgaccggtctggtacagggcgcccaggaaacggctcgcctgagcgcgattcagagcaactacgttggtaaagtcgctgatttgacagtgcctcagttaatcctgtcattcattcctaaaaacccgtttgccgacttaaccggtgccagcccgacgtccatcatcagcgtggtcatcttcgccgcgttcctgggtgtggctgccctgcagttgcacaaggatgatgaggtaaaaggccagcgggtactgaccgccattgatacgctgcagtcgtgggtgatgaagctggttcgcctgattatgaagctgacaccttatggtgtgctggcgctgatgaccaaagtcgttgccggctccaacgtgcaggacatcatcaagctgggcagctttgttgtggcatcctatttgggcctgacccttatgtttgtggttcatgcgctgctcctgtcggtcaacggcatcaatcctatgcgcttcttccgcaaagtgtggccggtattgacctttgcctttaccagccgctccagcgccgccagcattccactgaacgtggaggcgcaaacccgccgccttggcgtacctgaatcgattgccagcttctcggcctcatttggtgccaccattggacagaacggctgtgccggtctttatccgacgatgctggcagtgatggtcgccccgacggtcggcatcaatccttttgatccgctgtggattgccacgctggtcggcattgtgacattgagttcagcaggcgtagccggtgtcggcggcggagcgacctttgccgcgctgatcgtcctgcctgcgatggggctgccggtcacgctggtcgcgctgctgatttccattgagcctctgattgacatgggacgaacagcgttaaacgtcaatggctcaatgaccgcaggatcgctcaccagtcgctggctgggcctgaccgataaacgcgttttagagcgcagcgaacacagtgaattagagcacagctaa

SEQ ID NO: 4 - protein, 463 aa, Pantoea ananatis
MDIPLTLNVVAFVLLLLLLSRLGRANWSLSKKVLTGLVLGVVFGLALQLIYGGNSDIVKASIGWFNIVGNGYVQLLQMIVMPLVFVSILSAVARLHNASSLGKISFLTIGVLLFTTAISALIGVFVTGLFHLNATGLVQGAQETARLSAIQSNYVGKVADLTVPQLILSFIPKNPFADLTGASPTSIISVVIFAAFLGVAALQLHKDDEVKGQRVLTAIDTLQSWVMKLVRLIMKLTPYGVLALMTKVVAGSNVQDIIKLGSFVVASYLGLTLMFVVHALLLSVNGINPMRFFRKVWPVLTFAFTSRSSAASIPLNVEAQTRRLGVPESIASFSASFGATIGQNGCAGLYPTMLAVMVAPTVGINPFDPLWIATLVGIVTLSSAGVAGVGGGATFAALIVLPAMGLPVTLVALLISIEPLIDMGRTALNVNGSMTAGSLTSRWLGLTDKRVLERSEHSELEHS

SEQ ID NO: 5 - DNA, 801 bp, Escherichia coli
  CDS (1)..(801)
atgaaattagcacatctgggacgtcaggcattgatgggtgtgatggccgtggcgctggttgcgggcatgagcgttaaaagttttgcagatgaaggtctgcttaataaagttaaagagcgcggcacgctgctggtagggctggaaggaacttatccgccgttcagttttcagggagatgacggcaaattaaccggttttgaagtggaatttgcccaacagctggcaaaacatcttggcgttgaggcgtcactaaaaccgaccaaatgggacggtatgctggcgtcgctggactctaaacgtattgatgtggtgattaatcaggtcaccatttctgatgagcgcaagaaaaaatacgatttctcaaccccgtacaccatttctggtattcaggcgctggtgaaaaaaggtaacgaaggcaccattaaaacagccgatgatctgaaaggcaaaaaagtgggggtcggtctgggcaccaactatgaagagtggctgcggcagaatgttcagggcgtcgatgtgcgtacctatgatgatgacccgaccaaatatcaggatctgcgcgtagggcgtatcgatgcgatcctcgttgatcgtctggcggcgctggatctggtgaagaaaaccaacgatacgctggcagtaaccggtgaagcattctcccgtcaggagtctggcgtggcgctgcgtaaaggaaatgaggacctgctgaaagcagtgaatgatgcaattgcggaaatgcaaaaagatggcactctgcaagccctttccgaaaaatggtttggtgctgatgtgaccaaataa

SEQ ID NO: 6 - protein, 266 aa, Escherichia coli
MKLAHLGRQALMGVMAVALVAGMSVKSFADEGLLNKVKERGTLLVGLEGTYPPFSFQGDDGKLTGFEVEFAQQLAKHLGVEASLKPTKWDGMLASLDSKRIDVVINQVTISDERKKKYDFSTPYTISGIQALVKKGNEGTIKTADDLKGKKVGVGLGTNYEEWLRQNVQGVDVRTYDDDPTKYQDLRVGRIDAILVDRLAALDLVKKTNDTLAVTGEAFSRQESGVALRKGNEDLLKAVNDAIAEMQKDGTLQALSEKWFGADVTK

SEQ ID NO: 7 - DNA, 801 bp, Pantoea ananatis
  CDS (1)..(801)
atggctttctcacagattcgtcgccaggtggtgacgggaatgatggcggttgcgctggtggcaggcttcagcgttaaaacgtttgcggcagacgatttactggcgcaggttaaaagcaaaggcgagctgcgcgtcggtctggaaggcacctatcctcctttcagttttcaggatgaaaagggcaagctgacgggctttgaagtggagtttgctcaggatctggccaaacatatgggggttaaagccgtcctgaagccaaccaagtgggatggcatgttagcggcgctggattcgaaacgcatcgatgtggtcatcaatcaggtgacgatctccgatgagcgtaaaaagaaatatgatttctctacgccgtatacgatttctggcgtgcaggcgctgacgctgaagaaaaacgcgggcagcatcactaagccagaagacctcgcagggaagaaagtcggtgtgggcctggggaccaactacgagcagtggctgcgcgcgaacgtaaaaggtgtggatatccgcacttatgatgatgacccaaccaaatatcaggatctgcgttctggtcgcgttgatgcgattctggtggatcgcctggctgctctggatctggtgaagaaaacgggcgataccatggcggtagccggtccggcattctcgcgtctggaagcgggcgttgcgctgcgtaagggcaacgaagatttactgaaggcgatcgatcaggccattgcggaaatgcagaaagacggcacccttaaaaagctgtcagaaaaatggtttggcgcggacgttactaaataa

SEQ ID NO: 8 - protein, 266 aa, Pantoea ananatis
MAFSQIRRQVVTGMMAVALVAGFSVKTFAADDLLAQVKSKGELRVGLEGTYPPFSFQDEKGKLTGFEVEFAQDLAKHMGVKAVLKPTKWDGMLAALDSKRIDVVINQVTISDERKKKYDFSTPYTISGVQALTLKKNAGSITKPEDLAGKKVGVGLGTNYEQWLRANVKGVDIRTYDDDPTKYQDLRSGRVDAILVDRLAALDLVKKTGDTMAVAGPAFSRLEAGVALRKGNEDLLKAIDQAIAEMQKDGTLKKLSEKWFGADVTK

SEQ ID NO: 9 - DNA, 1422 bp, Escherichia coli
  CDS (301)..(1119)
tcggtcagggcatggatgtacaaagcgcgcaggagaagattggtcaggtggtggaaggctaccgcaatacgaaagaagtccgcgaactggcgcatcgcttcggcgttgaaatgccaataaccgaggaaatttatcaagtattatattgcggaaaaaacgcgcgcgaggcagcattgactttactaggtcgtgcacgcaaggacgagcgcagcagccactaaccccagggaacctttgttaccgctatgacccggcccgcgcagaacgggccggtcattatctcatcgtgtggagtaagcaatgtcgtgtgaagaactggaaattgtctggaacaatattaaagccgaagccagaacgctggcggactgtgagccaatgctggccagtttttaccacgcgacgctactcaagcacgaaaaccttggcagtgcactgagctacatgctggcgaacaagctgtcatcgccaattatgcctgctattgctatccgtgaagtggtggaagaagcctacgccgctgacccggaaatgatcgcctctgcggcctgtgatattcaggcggtgcgtacccgcgacccggcagtcgataaatactcaaccccgttgttatacctgaagggttttcatgccttgcaggcctatcgcatcggtcactggttgtggaatcaggggcgtcgcgcactggcaatctttctgcaaaaccaggtttctgtgacgttccaggtcgatattcacccggcagcaaaaattggtcgcggtatcatgcttgaccacgcgacaggcatcgtcgttggtgaaacggcggtgattgaaaacgacgtatcgattctgcaatctgtgacgcttggcggtacgggtaaatctggtggtgaccgtcacccgaaaattcgtgaaggtgtgatgattggcgcgggcgcgaaaatcctcggcaatattgaagttgggcgcggcgcgaagattggcgcaggttccgtggtgctgcaaccggtgccgccgcataccaccgccgctggcgttccggctcgtattgtcggtaaaccagacagcgataagccatcaatggatatggaccagcatttcaacggtattaaccatacatttgagtatggggatgggatctaatgtcctgtgatcgtgccggatgcgatgtaatcatctatccggcctacagtaactaatctctcaataccgctcccggataccccaactgccgccaggcttcatacaccactaccgacaccgcattggacagattcatgctgcggctgtccggcaccatcggaatgcgaattttttgttcagcgggcagggcatcaagaatgctcgctggcaggccgcgtgtttccgggccgaacatcagataatcgccatcctgatagcttacggcgctgtgagcaggtgtacctttcgtggtgagggcga

SEQ ID NO: 10 - protein, 273 aa, Escherichia coli
MSCEELEIVWNNIKAEARTLADCEPMLASFYHATLLKHENLGSALSYMLANKLSSPIMPAIAIREVVEEAYAADPEMIASAACDIQAVRTRDPAVDKYSTPLLYLKGFHALQAYRIGHWLWNQGRRALAIFLQNQVSVTFQVDIHPAAKIGRGIMLDHATGIVVGETAVIENDVSILQSVTLGGTGKSGGDRHPKIREGVMIGAGAKILGNIEVGRGAKIGAGSVVLQPVPPHTTAAGVPARIVGKPDSDKPSMDMDQHFNGINHTFEYGDGI

SEQ ID NO: 11 - DNA, 669 bp, Pantoea ananatis
  CDS (1)..(669)
atgcaggaaagtttacaactggtgctggactcagcaccctttcttcttaagggcgcgctgttcacgctgcagctcagtattggcggtatgtttttcgggctgatactgggctttttgctggcgctgatgcgcctgtcccgcttctggcccgtgaactggctggcgcgtatctacgtgtccatctttcgtggcacgccgcttatcgcccagctgtttatgatctactacggtctgccgcagtttggcattgagctggatcccattccctcggcgatgattgggctgtcgcttaacatggcggcctatgcctcagaatctctgcgcggcgcgattgctgccatcgagcgcggccagtgggaagcggcggccagtatcggcatgacgccgtggcaaacgctgcgtcgggttgttttaccgcaggccgcccgcaccgcactcccgcctttgggtaacagctttatcagtctggtaaaagatacctcgctggctgccacgatccaggttccggaactgtttcgtcaggcgcagctgattacttcacgcacgttggaggtgttcaccatgtatctggcggcttcgctgatctattgggtgatggcaaccgtactgtcagcattgcagaaccgactggaagcacacgttaaccgtcaggatcaggaggcgaaatga

SEQ ID NO: 12 - protein, 222 aa, Pantoea ananatis
MQESLQLVLDSAPFLLKGALFTLQLSIGGMFFGLILGFLLALMRLSRFWPVNWLARIYVSIFRGTPLIAQLFMIYYGLPQFGIELDPIPSAMIGLSLNMAAYASESLRGAIAAIERGQWEAAASIGMTPWQTLRRVVLPQAARTALPPLGNSFISLVKDTSLAATIQVPELFRQAQLITSRTLEVFTMYLAASLIYWVMATVLSALQNRLEAHVNRQDQEAK

SEQ ID NO: 13 - DNA, 783 bp, Pantoea ananatis
  CDS (1)..(753)
atgagtgctattgaagttcgccagttggtgaaaaagtttaacggacaaacggtactgcacggcatcgatctcgatgtcgccccgggcgaaatcgtcgcgataatcggccccagtggctcaggtaaaaccacgctgttgcgcagcatcaacctccttgaggtcccggatgctggccgcattaaagtgggtgacatcaccattgacgccagcctgggcatgaacagacagaaagagcgggtgcggatgttgcgtcagcaggtgggttttgtctttcaaaacttcaatttatttccgcatcgttcggtgctggaaaatattattgaaggtccggtgattgtgaagcgggaagcgaaagcggacgcggttgcccgcgcgcgcagcctgcttgaaaaagttggactcaacggcaaggaagacagctacccacggcgcctgtccggtggtcagcagcagcgtgtcgccattgcccgtgcgttggcgatgcagccagaagtcattttgtttgatgaaccgacctctgcgctggatccggaactggtgggtgaagtactgaacaccattcgcggactggctcaggaaaaacgcaccatggttatcgtgacgcacgagatgagctttgcccgcgacgtcgccgaccgcgccatttttatggatcagggccgtgttgttgaacagggccctgctaaggcactcttcagcgatcctcaggagccgcgtacccggcagtttctgaataaatttcttaatcaataaacccggtgccgccagaagttggcggcgtaa

SEQ ID NO: 14 - protein, 250 aa, Pantoea ananatis
MSAIEVRQLVKKFNGQTVLHGIDLDVAPGEIVAIIGPSGSGKTTLLRSINLLEVPDAGRIKVGDITIDASLGMNRQKERVRMLRQQVGFVFQNFNLFPHRSVLENIIEGPVIVKREAKADAVARARSLLEKVGLNGKEDSYPRRLSGGQQQRVAIARALAMQPEVILFDEPTSALDPELVGEVLNTIRGLAQEKRTMVIVTHEMSFARDVADRAIFMDQGRVVEQGPAKALFSDPQEPRTRQFLNKFLNQ

SEQ ID NO: 15 - DNA, 1039 bp, Escherichia coli
  CDS (201)..(839)
tgcggataacggtagaatttttacgccagtattttgccgagcactacccgaatttttcactggagcatgcctgattaatgattcaattatcgggttgatatcaggttaaaacctgattttctcctttctaagccgctacagattggttagcatattcacctttaatcgcgcatgatcgaaagataattaaagaggttaatgtgttcgctgaatacggggttctgaattactggacctatctggttggggccatttttattgtgttggtgccagggccaaataccctgtttgtactcaaaaatagcgtcagtagcggtatgaaaggcggttatcttgcggcctgcggtgtatttattggcgatgcggtattgatgtttctggcatgggctggagtggcgacattaattaagaccaccccgatattattcaacattgtacgttatcttggtgcgttttatttgctctatctggggagtaaaattctttacgcgaccctgaagggtaaaaatagcgaggccaaatccgatgagccccaatacggtgctatttttaaacgcgcgttaattttgagcctgactaatccgaaagccattttgttctatgtgtcgtttttcgtacagtttatcgatgttaatgccccacatacgggaatttcattctttattctggcggcgacgctggaactggtgagtttctgctatttgagcttcctgattatatctggtgcttttgtcacgcagtacatacgtaccaaaaagaaactggctaaagttggcaactcactgattggtttgatgttcgtgggtttcgctgcccgactggcgacgctgcaatcctgatgctttcagcccgcgttgtcgcgggcttcccatctataatcctccctgattcttcgctgatatggtgctaaaaagtaaccaataaatggtatttaaaatgcaaattatcaggcgtaccctgaaacggctggaataaaccgttttcagcgcattcaccgaaggagggaaaaggatgcttcaaatcccacagaattatattc

SEQ ID NO: 16 - protein, 212 aa, Escherichia coli [sequence omitted]

SEQ ID NO: 17 - DNA, 1779 bp, Pantoea ananatis
  CDS (301)..(1536)
gtcaaaaccctcaaaaaataaagcaccgggcgcacaacagggcgcccgctttttttgatttaaaaaaacttttctcaccaggctgaaatttggtgacttatgtcacataaccgtcatcggcagcgggttcgttcttctcgatgcggccaacccacgattttgtctggcaaagtacgtcctctgagccctgccatgctggcggtcaggcaatcgtttgtattgccgcaggcgatttttttgatattttgacagacggctgactgcgttcagtcctcgttgaattctgaatagggttgggaaatggcaaaggtatcactggaaaaagacaaaattaagttcctgctggtggaaggtgtccatcagagcgcgctggaaaatcttcgtgctgcaggttacaccaatattgaattccacaaaggcgcactggatgccgaggcgttaaaagcttccgctcgcgatgcgcattttatcggtatccgttcccgttcccaactgaccgaagagatttttgccgctgcagaaaaactggtagcggtgggctgtttctgtatcggaacgaatcaggttgatttaaatgccgcagcgaaacgcggtatcccggtttttaacgcacctttctcaaatacgcgctctgtggccgagctggttattggcgagatgctgctgatgctgcgcggtgttccggaagcgaatgccaaagcgcaccgtggtatctggaataaaatcgccaaaggctcttttgaagcgcgcggtaaaaagctgggtatcattggctatggccatatcggtatgcaactgggcgtgctggcagaaagtctgggcatgcacgtttacttctatgacatcgaaaacaagctgccgttgggcaacgcatcacaggttcgtagcctgacgcagttgctaaatatgagtgacgttgtcagcctgcatgtcccggaaaccgcctctacgcaaaatatgatttctgccaatgagctggctcagatgaagcctggcggcctgctgataaatgcctcacgcggcaccgtggtagatattcctgctttgtgcgaagcgctggccagcaagcaggttggtggcgctgcgattgatgtgttccctgtagagccggcgaccaacagcgatccgtttgtttccccactgagcgaattcgacaacgttatcctgacgccgcacatcgggggatcgacggaagaagctcaggagaatatcgggattgaagtcgcgggcaagctggcgaaatattcggataacggttcaacgctgtccgccgtcaatttcccggaagtgtcattgccgatgcacggcattagcgccagtcgtctgctgcatattcacgaaaaccgtccgggcgttctcaccgcgatcaaccagattttcgctgaacaaggcatcaacattgccgctcagtacctgcaaacctctccgatgatgggttatgtggtcatcgacattgatgctgagcacgaactggcagagaaagctctgcaactgatgaaggcgattccgggaacgattcgcgcccgcctgctttactgatcccacgctgtcacctacccgggcacacaagcatgcccgggtttattcatcccatagccacagttttgatggcgtcagcacggccggcaaaggaatgtcccacgccgctgtaggcagcgcgtcaacccgctgacagtcatgagcgatgcccaccggtaaaaacccatgctgtttccagttctgtaaggtgcgatcgtagaagccgccccccattcctaaacgctgtccggcgcgatcgaa

SEQ ID NO: 18 - protein, 412 aa, Pantoea ananatis
MAKVSLEKDKIKFLLVEGVHQSALENLRAAGYTNIEFHKGALDAEALKASARDAHFIGIRSRSQLTEEIFAAAEKLVAVGCFCIGTNQVDLNAAAKRGIPVFNAPFSNTRSVAELVIGEMLLMLRGVPEANAKAHRGIWNKIAKGSFEARGKKLGIIGYGHIGMQLGVLAESLGMHVYFYDIENKLPLGNASQVRSLTQLLNMSDVVSLHVPETASTQNMISANELAQMKPGGLLINASRGTVVDIPALCEALASKQVGGAAIDVFPVEPATNSDPFVSPLSEFDNVILTPHIGGSTEEAQENIGIEVAGKLAKYSDNGSTLSAVNFPEVSLPMHGISASRLLHIHENRPGVLTAINQIFAEQGINIAAQYLQTSPMMGYVVIDIDAEHELAEKALQLMKAIPGTIRARLLY

SEQ ID NO: 19 - DNA, 4403 bp, Pantoea ananatis
  CDS (301)..(1311)
  CDS (1317)..(2147)
  CDS (2150)..(3022)
tacagcggaacctggcacgggccagaagggttgatgccgtcggatgacacactcaagagctggacgctcagcaaaattgtctggcagcgctaagtcttttttcacaccgctcaaccgcagggcataaccggccctgcgcgtccaattctgtttttcgtctgtcttttcccgccgccttatgcctttttcgactttgaaatcagcaaacgatatataaaaccgttacgggtttacgctgagttataaataaactgctgtatctgcagatgagatctgcatcaaatttcctcagggtgaaccatgaccttaccagcgatgaaaaaaatcgtgagcggactcgcactgtcgctgagtctggccggtgccgcaaacgcgaccgagctgttgaacagctcttacgatgtcgcacgtgaattatttgtcgccctgaatgcgccttttgtcagccagtgggatgccagccatcctgacgacaagctgaccattaagatgtcccatgccgggtcatccaaacaggcgctggcgatcctgcaaggcctgcgtgccgatgtggtgacctataaccaggtcaccgatgtgcaggtgctgcacgataaaggcaaactgatccctgccgactggcaaacccgcctgccgaataacagttcgccgttttactccaccatggcgttcctggtgcgcaagggaaacccaaagcagattcacgactggtccgatttaacccgtgacgatgtgaagctgatttttcctaatcccaaaacctcgggcaacggacgttatacctatcttgctgcctggggcgccgccagcaacactgacgggggcgatcaggctaaaacccgcgcttttatgacaaaatttctgaaaaatgttgaagtcttcgataccggtggccgaggtgctacgaccacctttgctgaacgcggtctgggcgatgtgttgatcagttttgagtctgaagtgaataacatccgcaaccagtacggcaaagacgactacgaagtcgtggtgcctaaaaccgatattctcgcggagtttcccgttgcctgggtagataaaaacgtcgagcagaataaaacagccgatgcagcgaaagcctatctgacctggctgtattctcctgcggcgcagaaaattattacggatttctattaccgcgtgaacaatccgcagttaatggcgcagcaaaaagcccgttttcctgccacgaacctgtttcgtgttgaagacatttttggcggctgggataacgtgatgaaaacccatttcgccagcggtggcgagctagaccagttattagcggcggggcggtgatcatgtttgcagccagccaaaaacgcgtcctgcccggtttcggtctcagcctgggcaccagcctgctctttacctgtctggtgctgctgctgccaatcagcgcactgattatgcagctgtcgcagatgacgttgcagcaatactgggacgtggtcaccaatccgcagctcatcgcggcctataaggtcacgctgctgtcggccggtgtggcctcactgtttaatgccgtattcggcatgttaatggcgtggatcttaacgcgttaccgttttccgggccgcacgctgctcgatggtctgatggatctgccgtttgcgctgccgaccgcggttgctggcctgacgctggccggtctgttttccgtgaacggctggtacggacaatggttcgcgcattttgatatcaagatctcctatacctggatcggtatcgcgctcgcgatggccttcaccagtattccgtttgtggtgcgtaccgtgcagccggtgctggaagagctggggcctgaatatgaggaagcggctcaaacgctgggcgccacgccctggcagagcttccgccgggtcgttctgcctgaagtggcaccggccttacttgcgggcaccgcgctgtcgtttacccgcagcctgggcgagtttggtgcggtaatctttattgccggcaacatcgcttggaaaaccgaagtgacctcgctgatgatcttcgtgcgcctgcaggagtttgactatccggcagccagcgccattgcctcggtcattctggcggcatcactgctgttacttttcgctatcaataccttacaaagccgctttggtcgtcgtctgggaggccattaatggcagagatttcgcaactcaatcatgccgaccgccagcctgttaactgggccaagtggctgcttattggtattggtgcgctgatatccttgctgctgctggtcgtgccgatggtgtccatcttctgggaggccctgcataaaggactgggcgtcaccttaagtaatctgaccgacagcgacatgctccatgccatatggctcacggtgctggtcgcattgattaccgtgccggtgaatttagtgttcggcacgctgctggcctggctggtgacacgctttacctttccgggacgtcagctgcttttgacgctgttcgatattccctttgcggtatcgcctgtggtcgccggtctgatgtatctcctgttctggggcattaacggcccggcgggcggctggctggatgcccataatattcaggtgatgttctcctggcctggcatggtgctggtcaccgtcttcgttacctgtccgtttgtggtgcgcgaactggtgccggtgatgctgagccagggcagtcatgaagatgaagccgcggtgctgttaggtgcctcgggctggcagatgttccgtcgcgtgacgctgccgaatattcgctgggccatgctgtatggcgtcgtgctgaccaacgcccgcgcgattggtgagtttggcgcggtttccgtggtttcgggttctattcgcggtgaaacctacactttaccgcttcaggttgaattactgcatcaggattacaacacggtgggcgcctttactgccgcagccttactgaccgtgatggcaatcgtgacgctgtttctgaaaagcattgtgcaatggcgtttagagcaacagcacaaacgcctgcaactggaggacaatcatgagcattgagattaaccagatcaacaaatcctttggtcgcacagcggtgctgaacgatatctcactggatattccttctggccagatggtggccttactggggccgtccggttccggtaaaaccacgctgctgcgcatcattgctggactggaacatcagaacagcggtcagattcgttttcacgaccacgatgtcagccgcctgcacgcccgcgatcgccaggtcggatttgtcttccagcactatgcgctgttccgtcatatgacggtcttcgacaatattgcctttggcctgaccgtgctgccgcgccgtgagcgtccgtccagtgcggaaattaaaaaacgcgtcacgcgcctgctggagatggtgcagctttcccatctggcgaaccgtttcccggcccagctttcgggagggcagaagcagcgcgtcgcgctggcaagagccctggccgtggaaccgcaaatcctgttgctggatgagccctttggtgcgctggacgctcaggtgcgtaaagagctgcgccgttggttacgtcagctgcacgaagaattgaagttcaccagcgtgttcgtcacccacgatcaggaagaggcgatggaagtggccgatcgcgtggtggtgatgagccagggcagcatcgaacaggtggggacgccggatgaagtctggcgcgatcccgccacgcgcttcgtgctggaattcctgggtgaggttaaccgcttcgacggtgaagtgcatggttctcagttccatgtcggggcgcaccactggccgttaggctatacctctgcacatcagggcgcggtcgatctgttcctgcgcccgtgggaaatcgacgtttcgcgcagaagtagcctggaaacgccgctgcccgttcaggtcttagaagtgagtcctcgtggtcacttctggcagctggtggtgcagccaacgggatggcagagcgagcccttctcgctggtctttgacggtgaacagaccgcgccgttgcgcggcgagcgcctgttcgtggggctgcagcaggccagactgtaccagggcgcgacaccgttacgggcggttgcctttgcacacagcgcctgataggttgagtgaatgttaaacgcccggaggcgcttcccgcgatccgggctttttaatggcaaggtttgtaacctgtagacctgataagacgcgcaagcgtcgcatcaggcaacaccacgtatggatagagatcgtgagtacattagaacaaacaataggcaatacgcctctggtgaagttgcagcgaatggggccggataacggcagtgaagtgtggttaaaactggaaggcaataacccggcaggttcggtgaaagatcgtgcggcactttcgatgatcgtcgaggcggaaaagcgcgg

SEQ ID NO: 20 - protein, 337 aa, Pantoea ananatis
MTLPAMKKIVSGLALSLSLAGAANATELLNSSYDVARELFVALNAPFVSQWDASHPDDKLTIKMSHAGSSKQALAILQGLRADVVTYNQVTDVQVLHDKGKLIPADWQTRLPNNSSPFYSTMAFLVRKGNPKQIHDWSDLTRDDVKLIFPNPKTSGNGRYTYLAAWGAASNTDGGDQAKTRAFMTKFLKNVEVFDTGGRGATTTFAERGLGDVLISFESEVNNIRNQYGKDDYEVVVPKTDILAEFPVAWVDKNVEQNKTADAAKAYLTWLYSPAAQKIITDFYYRVNNPQLMAQQKARFPATNLFRVEDIFGGWDNVMKTHFASGGELDQLLAAGR

SEQ ID NO: 21 - protein, 277 aa, Pantoea ananatis
MFAASQKRVLPGFGLSLGTSLLFTCLVLLLPISALIMQLSQMTLQQYWDVVTNPQLIAAYKVTLLSAGVASLFNAVFGMLMAWILTRYRFPGRTLLDGLMDLPFALPTAVAGLTLAGLFSVNGWYGQWFAHFDIKISYTWIGIALAMAFTSIPFVVRTVQPVLEELGPEYEEAAQTLGATPWQSFRRVVLPEVAPALLAGTALSFTRSLGEFGAVIFIAGNIAWKTEVTSLMIFVRLQEFDYPAASAIASVILAASLLLLFAINTLQSRFGRRLGGH

SEQ ID NO: 22 - protein, 291 aa, Pantoea ananatis
MAEISQLNHADRQPVNWAKWLLIGIGALISLLLLVVPMVSIFWEALHKGLGVTLSNLTDSDMLHAIWLTVLVALITVPVNLVFGTLLAWLVTRFTFPGRQLLLTLFDIPFAVSPVVAGLMYLLFWGINGPAGGWLDAHNIQVMFSWPGMVLVTVFVTCPFVVRELVPVMLSQGSHEDEAAVLLGASGWQMFRRVTLPNIRWAMLYGVVLTNARAIGEFGAVSVVSGSIRGETYTLPLQVELLHQDYNTVGAFTAAALLTVMAIVTLFLKSIVQWRLEQQHKRLQLEDNHEH

SEQ ID NO: 23 - DNA, 1403 bp, Pantoea ananatis
  CDS (15)..(1103)
actggaggacaatcatgagcattgagattaaccagatcaacaaatcctttggtcgcacagcggtgctgaacgatatctcactggatattccttctggccagatggtggccttactggggccgtccggttccggtaaaaccacgctgctgcgcatcattgctggactggaacatcagaacagcggtcagattcgttttcacgaccacgatgtcagccgcctgcacgcccgcgatcgccaggtcggatttgtcttccagcactatgcgctgttccgtcatatgacggtcttcgacaatattgcctttggcctgaccgtgctgccgcgccgtgagcgtccgtccagtgcggaaattaaaaaacgcgtcacgcgcctgctggagatggtgcagctttcccatctggcgaaccgtttcccggcccagctttcgggagggcagaagcagcgcgtcgcgctggcaagagccctggccgtggaaccgcaaatcctgttgctggatgagccctttggtgcgctggacgctcaggtgcgtaaagagctgcgccgttggttacgtcagctgcacgaagaattgaagttcaccagcgtgttcgtcacccacgatcaggaagaggcgatggaagtggccgatcgcgtggtggtgatgagccagggcagcatcgaacaggtggggacgccggatgaagtctggcgcgatcccgccacgcgcttcgtgctggaattcctgggtgaggttaaccgcttcgacggtgaagtgcatggttctcagttccatgtcggggcgcaccactggccgttaggctatacctctgcacatcagggcgcggtcgatctgttcctgcgcccgtgggaaatcgacgtttcgcgcagaagtagcctggaaacgccgctgcccgttcaggtcttagaagtgagtcctcgtggtcacttctggcagctggtggtgcagccaacgggatggcagagcgagcccttctcgctggtctttgacggtgaacagaccgcgccgttgcgcggcgagcgcctgttcgtggggctgcagcaggccagactgtaccagggcgcgacaccgttacgggcggttgcctttgcacacagcgcctgataggttgagtgaatgttaaacgcccggaggcgcttcccgcgatccgggctttttaatggcaaggtttgtaacctgtagacctgataagacgcgcaagcgtcgcatcaggcaacaccacgtatggatagagatcgtgagtacattagaacaaacaataggcaatacgcctctggtgaagttgcagcgaatggggccggataacggcagtgaagtgtggttaaaactggaaggcaataacccggcaggttcggtgaaagatcgtgcggcactttcgatgatcgtcgaggcggaaaagcgcgg

SEQ ID NO: 24 - protein, 362 aa, Pantoea ananatis
MSIEINQINKSFGRTAVLNDISLDIPSGQMVALLGPSGSGKTTLLRIIAGLEHQNSGQIRFHDHDVSRLHARDRQVGFVFQHYALFRHMTVFDNIAFGLTVLPRRERPSSAEIKKRVTRLLEMVQLSHLANRFPAQLSGGQKQRVALARALAVEPQILLLDEPFGALDAQVRKELRRWLRQLHEELKFTSVFVTHDQEEAMEVADRVVVMSQGSIEQVGTPDEVWRDPATRFVLEFLGEVNRFDGEVHGSQFHVGAHHWPLGYTSAHQGAVDLFLRPWEIDVSRRSSLETPLPVQVLEVSPRGHFWQLVVQPTGWQSEPFSLVFDGEQTAPLRGERLFVGLQQARLYQGATPLRAVAFAHSA

SEQ ID NO: 25 - DNA, 1512 bp, Escherichia coli
  CDS (301)..(1212)
agccgctggggtggtacaacgaaccgctgacggtcgtgatgcatggcgacgatgccccgcagcgtggcgagcgtttattcgttggtctgcaacatgcgcggctgtataacggcgacgagcgtatcgaaacccgcgatgaggaacttgctctcgcacaaagcgcctgataggttgagtgaatgttaaacgcccggaggcgcttcccgcgatccgggctttttaatggcaaggtttgtaacctgtagacctgataagacgcgcaagcgtcgcatcaggcaacaccacgtatggatagagatcgtgagtacattagaacaaacaataggcaatacgcctctggtgaagttgcagcgaatggggccggataacggcagtgaagtgtggttaaaactggaaggcaataacccggcaggttcggtgaaagatcgtgcggcactttcgatgatcgtcgaggcggaaaagcgcggggaaattaaaccgggtgatgtcttaatcgaagccaccagtggtaacaccggcattgcgctggcaatgattgccgcgctgaaaggctatcgcatgaaattgctgatgcccgacaacatgagccaggaacgccgtgcggcgatgcgtgcttatggtgcggaactgattcttgtcaccaaagagcagggcatggaaggtgcgcgcgatctggcgctggagatggcgaatcgtggcgaaggaaagctgctcgatcagttcaataatcccgataacccttatgcgcattacaccaccactgggccggaaatctggcagcaaaccggcgggcgcatcactcattttgtctccagcatggggacgaccggcactatcaccggcgtctcacgctttatgcgcgaacaatccaaaccggtgaccattgtcggcctgcaaccggaagagggcagcagcattcccggcattcgccgctggcctacggaatatctgccggggattttcaacgcttctctggtggatgaggtgctggatattcatcagcgcgatgcggaaaacaccatgcgcgaactggcggtgcgggaaggaatattctgtggcgtcagctccggcggcgcggttgccggagcactgcgggtggcaaaagctaaccctgacgcggtggtggtggcgatcatctgcgatcgtggcgatcgctacctttctaccggggtgtttggggaagagcattttagccagggggcggggatttaaggattaatagcatcggagactgatgacaaacgcaaaactgcctgatgcgctacgcttatcaggcctacaaggtttctgcaatatattgaattagcacgattttgtaggccggataaggcgtttacgccgcatccggcataaacaaagcgcacttttttaacagttgttgctgccgacaaatgcagtatttaattttcgtgaggaaacgccgtaaggtcatcaatcattttttgaagtattggtgagtcctgaccgtcaccccattgaagaatttttgcggtaagctgatgacgcgctagt

SEQ ID NO: 26 - protein, 303 aa, Escherichia coli
VSTLEQTIGNTPLVKLQRMGPDNGSEVWLKLEGNNPAGSVKDRAALSMIVEAEKRGEIKPGDVLIEATSGNTGIALAMIAALKGYRMKLLMPDNMSQERRAAMRAYGAELILVTKEQGMEGARDLALEMANRGEGKLLDQFNNPDNPYAHYTTTGPEIWQQTGGRITHFVSSMGTTGTITGVSRFMREQSKPVTIVGLQPEEGSSIPGIRRWPTEYLPGIFNASLVDEVLDIHQRDAENTMRELAVREGIFCGVSSGGAVAGALRVAKANPDAVVVAIICDRGDRYLSTGVFGEEHFSQGAGI

SEQ ID NO: 27 - DNA, 313 bp, Escherichia coli
gcatgcttccaactgcgctaatgacgcagctggacgaaggcgggattctcgtcttacccgtaggggaggagcaccagtatttgaaacgggtgcgtcgtcggggaggcgaatttattatcgataccgtggaggccgtgcgctttgtccctttagtgaagggtgagctggcttaaaacgtgaggaaatacctggatttttcctggttattttgccgcaggtcagcgtatcgtgaacatcttttccagtgttcagtagggtgccttgcacggtaattatgtcactggttattaaccaatttttcctgggggtcgac

SEQ ID NO: 28 - DNA, 312 bp, Artificial Sequence
  mutant nlpD promoter
gcatgcttccaactgcgctaatgacgcagctggacgaaggcgggattctcgtcttacccgtaggggaggagcaccagtatttgaaacgggtgcgtcgtcggggaggcgaatttattatcgataccgtggaggccgtgcgctttgtccctttagtgaagggtgagctggcttaaaacgtgaggaaatacctggatttttcctggttattttgccgcaggtcagcgtataatgaagatcttttccagtgttgacaagggtccttgcacggttataatgtcactggttattaaccaatttttcctgggggtcgac

SEQ ID NO: 29 - DNA, 313 bp, Artificial Sequence
  mutant nlpD promoter
gcatgcttccaactgcgctaatgacgcagctggacgaaggcgggattctcgtcttacccgtaggggaggagcaccagtatttgaaacgggtgcgtcgtcggggaggcgaatttattatcgataccgtggaggccgtgcgctttgtccctttagtgaagggtgagctggcttaaaacgtgaggaaatacctggatttttcctggttattttgccgcaggtcagcgtataatgaagatcttttccagtgttcagtagggtgccttgcacggttataatgtcactggttattaaccaatttttcctgggggtcgac

SEQ ID NO: 30 - DNA, 36 bp, Artificial Sequence
  primer P1
agctgagtcgacccccaggaaaaattggttaataac

SEQ ID NO: 31 - DNA, 33 bp, Artificial Sequence
  primer P2
agctgagcatgcttccaactgcgctaatgacgc

SEQ ID NO: 32 - DNA, 33 bp, Artificial Sequence
  primer P3
agctgatctagaaaacagaatttgcctggcggc

SEQ ID NO: 33 - DNA, 33 bp, Artificial Sequence
  primer P4
agctgaggatccaggaagagtttgtagaaacgc

SEQ ID NO: 34 - DNA, 32 bp, Artificial Sequence
  primer P5
agctgagtcgacgtgttcgctgaatacggggt

SEQ ID NO: 35 - DNA, 32 bp, Artificial Sequence
  primer P6
agctgatctagagaaagcatcaggattgcagc

SEQ ID NO: 36 - DNA, 59 bp, Artificial Sequence
  primer P7
atcgtgaagatcttttccagtgttnannagggtgccttgcacggtnatnangtcactgg

SEQ ID NO: 37 - DNA, 32 bp, Artificial Sequence
  primer P8
tggaaaagatcttcannnnncgctgacctgcg

SEQ ID NO: 38 - DNA, 60 bp, Artificial Sequence
  primer P9
tccgctcacgatttttttcatcgctggtaaggtcatttatcccccaggaaaaattggtta

SEQ ID NO: 39 - DNA, 63 bp, Artificial Sequence
  primer P10
tttcacaccgctcaaccgcagggcataaccggcccttgaagcctgcttttttatactaagttg

SEQ ID NO: 40 - DNA, 20 bp, Artificial Sequence
  primer P11
ctttgtccctttagtgaagg

SEQ ID NO: 41 - DNA, 44 bp, Artificial Sequence
  primer P12
agctgatctagaagctgactcgagttaatggcctcccagacgac

SEQ ID NO: 42 - DNA, 33 bp, Artificial Sequence
  primer P13
agctgagtcgacatggcaaaggtatcactggaa

SEQ ID NO: 43 - DNA, 34 bp, Artificial Sequence
  primer P14
gagaacgcccgggcgggcttcgtgaatatgcagc

SEQ ID NO: 44 - DNA, 32 bp, Artificial Sequence
  primer P15
agctgatctagacgtgggatcagtaaagcagg

SEQ ID NO: 45 - DNA, 22 bp, Artificial Sequence
  primer P16
aaaaccgcccgggcgttctcac

SEQ ID NO: 46 - DNA, 80 bp, Artificial Sequence
  primer DydjN(Pa)-F
acctctgctgctctcctgaccagggaatgctgcattacatcggagttgcttgaagcctgcttttttatactaagttggca

SEQ ID NO: 47 - DNA, 80 bp, Artificial Sequence
  primer DydjN(Pa)-R
agacaaaaacagagagaaagacctggcggtgtacgccaggtctggcgtgacgctcaagttagtataaaaaagctgaacga

SEQ ID NO: 48 - DNA, 80 bp, Artificial Sequence
  primer DfliY-FW
atggctttctcacagattcgtcgccaggtggtgacgggaatgatggcggttgaagcctgcttttttatactaagttggca

SEQ ID NO: 49 - DNA, 80 bp, Artificial Sequence
  primer DyecC-RV
ttacgccgccaacttctggcggcaccgggtttattgattaagaaatttatcgctcaagttagtataaaaaagctgaacga

SEQ ID NO: 50 - DNA, 80 bp, Artificial Sequence
  primer DcysE(Ec)-F
ccggcccgcgcagaacgggccggtcattatctcatcgtgtggagtaagcatgaagcctgcttttttatactaagttggca

SEQ ID NO: 51 - DNA, 80 bp, Artificial Sequence
  primer DcysE(Ec)-R
actgtaggccggatagatgattacatcgcatccggcacgatcacaggacacgctcaagttagtataaaaaagctgaacga

SEQ ID NO: 52 - DNA, 80 bp, Artificial Sequence
  primer DydjN(Ec)-F
cactatgactgctacgcagtgatagaaataataagatcaggagaacggggtgaagcctgcttttttatactaagttggca

SEQ ID NO: 53 - DNA, 80 bp, Artificial Sequence
  primer DydjN(Ec)-R
aaagtaaggcaacggcccctatacaaaacggaccgttgccagcataagaacgctcaagttagtataaaaaagctgaacga

SEQ ID NO: 54 - DNA, 43 bp, Artificial Sequence
  primer ydjN(Ec)-SalIFW2
acgcgtcgacatgaactttccattaattgcgaacatcgtggtg

SEQ ID NO: 55 - DNA, 35 bp, Artificial Sequence
  primer ydjN(Ec)-xbaIRV2
ctagtctagattaatggtgtgccagttcggcgtcg

SEQ ID NO: 56 - DNA, 29 bp, Artificial Sequence
  primer ydjN2(Pa)-SalIFW
acgcgtcgacatggatattcctcttacgc

SEQ ID NO: 57 - DNA, 29 bp, Artificial Sequence
  primer ydjN2(Pa)-xbaIRV
tgctctagattagctgtgctctaattcac

SEQ ID NO: 58 - DNA, 80 bp, Artificial Sequence
  primer DfliY(Ec)-FW
atgaaattagcacatctgggacgtcaggcattgatgggtgtgatggccgttgaagcctgcttttttatactaagttggca

SEQ ID NO: 59 - DNA, 80 bp, Artificial Sequence
  primer DfliY(Ec)-RV
ttatttggtcacatcagcaccaaaccatttttcggaaagggcttgcagagcgctcaagttagtataaaaaagctgaacga

SEQ ID NO: 60 - DNA, 33 bp, Artificial Sequence
  primer fliY(Ec)SalI-F
acgcgtcgacatgaaattagcacatctgggacg

SEQ ID NO: 61 - DNA, 31 bp, Artificial Sequence
  primer fliY(Ec)XbaI-R
ctagtctagattatttggtcacatcagcacc

SEQ ID NO: 62 - DNA, 147 bp, Escherichia coli
aaaacgtgaggaaatacctggatttttcctggttattttgccgcaggtcagcgtatcgtgaagatcttttccagtgttcagtagggtgccttgcacggtaattatgtcactggttattaaccaatttttcctgggggataaatgagc